(19) United States
(12) Patent Application Publication
(10) Pub. No.: US 2008/0214552 A1
(43) Pub. Date: Sep. 4, 2008

(54) Substituted Pyridazinecarboxamides and Derivatives Thereof

(76) Inventors: Rudiger Fischer, Pulheim (DE); Olga Malsam, Rosrath (DE); Gerhard Krautstrunk, Monheim (DE); Wolfgang Schaper, Diedorf (DE); Christian Arnold, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/571,103

(22) PCT Filed: Jun. 16, 2005
(86) PCT No.: PCT/EP2005/006462

§ 371 Date: Nov. 20, 2007

(30) Foreign Application Priority Data
Jun. 29, 2004 (DE) 10 2004 031 323.7

(51) Int. Cl. A01N 43/58; C07D 237/10; C07D 403/12; A01P 7/00; A01P 15/00

(52) U.S. Cl. 514/252.01; 544/224; 514/247; 544/238

(57) ABSTRACT

The present invention relates to compounds of the formula (I)

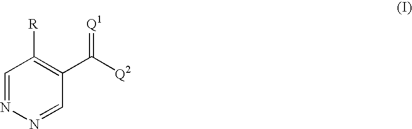

in which the substituents are as defined in the description, to the use of these compounds as plant treatment agents and pesticides, and to various processes for their preparation. Furthermore, the invention relates to novel intermediates for preparing compounds of the formula (I).

The present application of an invention relates to novel substituted pyridazinecarboxamides and derivatives thereof, to processes for their preparation and to their use as plant treatment agents and pesticides, in particular as insecticides.

A number of substituted pyridazinecarboxamides, such as, for example, the compounds N-benzyl-5-(1-hydroxyethyl)pyridazine-4-carboxamide and N-(t-butyl)-5-(1-hydroxyethyl)-pyridazine-4-carboxamide (cf. J. Heterocycl. Chem. 32 (1995), 841-846) and the compound N-[3,5-bis(trifluoromethyl)phenyl]-6-chloro-4-(trifluoromethyl)pyridazine-3-carboxamide (cf. J. Med. Chem. 43 (2000), 3995-4004) are already known from the literature. However, hitherto a possible use of these compounds for controlling animal pests has not been disclosed.

The present invention now provides novel substituted pyridazinecarboxamides and derivatives thereof of the formula (I),

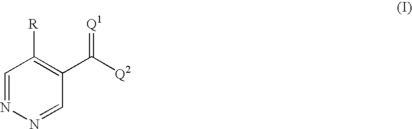

in which
R represents halogen-substituted alkyl,
Q¹ represents O (oxygen), S (sulfur) or the grouping N—R¹,
Q² represents one of the groupings below

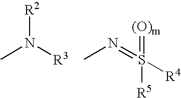

R¹ represents hydrogen, hydroxyl, or in each case optionally substituted alkyl, alkylcarbonyl, alkoxy, alkoxycarbonyl, alkylcarbonyloxy, alkoxycarbonyloxy, alkylsulfonyl, alkylsulfonyloxy, alkenyl, alkynyl, alkenyloxy, alkynyloxy, cycloalkyl, cycloalkyloxy, cycloalkylalkyl, cycloalkylalkoxy, cycloalkylcarbonyl, cycloalkylsulfonyl, cycloalkylsulfonyloxy, aryl, aryloxy, arylalkyl, arylalkoxy, arylcarbonyl, arylsulfonyl, aryl-sulfonyloxy, heterocyclyl, heterocyclylalkyl, heterocyclylcarbonyl, heterocyclylsulfonyl or heterocyclylsulfonyloxy,
R² represents one of the groupings -A¹-R⁶ or -A²-O—R⁶— or -A¹-S(O)ₙ—R⁶ or -A²-N(R⁶)₂ or -A²-N(R⁶)—O—R⁶ or -A²-O—N(R⁶)₂ or -A²-O—N═C(R⁶)₂ or -A²-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A²-(C=Q³)-N(R⁶)₂ or -A²-(C=Q³)-N(R⁶)OR⁶ or -A²-O—(C=Q³)-R⁶ or -A²-N(R⁶)—(C=Q³)-R⁶ or -A²-C(R⁶)═N—O—R⁶ or -A²-C(R⁶)═N(R⁶)—N(R⁶)₂ or -A²-C(R⁶)═N(R⁶)—N(R⁶)—(C=Q³)-(Q³)-R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different, and
R³ represents one of the groupings -A¹-R⁶ or -A¹-O—R⁶— or -A¹-S(O)ₙ—R⁶ or -A-N(R⁶)₂ or -A²-N(R⁶)—O—R⁶ or -A²-O—N(R⁶)₂ or -A²-O—N═C(R⁶)₂ or -A-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —N(R⁶)-A¹-(C=Q³)-R⁶ or -A¹-N(R⁶)—(C=Q³)-R⁶ or -A¹-C(R⁶)═N—O—R⁶ or -A¹-C(R⁶)═N(R⁶)—N(R⁶)₂ or -A²-C(R⁶)═N(R⁶)—N(R⁶)—(C=Q³)-(Q³)ₘ-R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different, or
R³ and R² together represent the grouping ═CR⁷R⁸, or
R¹ and R² together with the nitrogen atom to which the two groups are attached form an optionally substituted and optionally benzo-fused heterocycle which optionally contains one to three further nitrogen atoms and/or one oxygen atom and/or one (SO)ₙ-grouping and optionally additionally one (C=Q¹) grouping, or
R³ and R² together represents the grouping ═P(X)₃, where X represents amino, alkyl, alkylamino, cycloalkyl, cycloalkylamino, phenyl or phenylamino and where the radicals X may in each case be identical or different, and
R⁴ represents in each case optionally substituted alkyl, alkoxy, alkylamino, dialkylamino, alkenyl, alkynyl, cycloalkyl, cycloalkylamino, N-alkyl-cycloalkylamino, cycloalkenyl, cycloalkylalkyl, aryl, arylamino, N-alkyl-arylamino, arylalkyl, arylalkylamino, N-alkyl-arylalkylamino, N-cycloalkyl-arylalkylamino, heterocyclyl or heterocyclylalkyl, and
R⁵ represents in each case optionally substituted alkyl, alkoxy, alkylamino, dialkylamino, alkenyl, alkynyl, cycloalkyl, cycloalkylamino, N-alkyl-cycloalkylamino, cycloalkenyl, cycloalkylalkyl, aryl, arylamino, N-alkyl-arylamino, arylalkyl, arylalkylamino, N-alkyl-arylalkylamino, N-cycloalkyl-arylalkylamino, heterocyclyl or heterocyclylalkyl, or together with R⁴ represents optionally substituted and/or benzo-fused alkanediyl (alkylene), alkyleneamino or alkylenediamino, each of which is optionally interrupted by O (oxygen) and/or N (nitrogen) and/or an S(O)ₙ grouping and/or a (C=Q¹)-grouping,
R⁶ represents hydrogen, hydroxyl, amino, cyano or in each case optionally substituted alkyl, alkenyl, alkynyl, cycloalkyl, cycloalkenyl, cycloalkylalkyl, cycloalkenylalkyl, aryl, arylalkyl, heterocyclyl or heterocyclylalkyl, or—if two groupings R⁶ are located in a geminal position on a C═C double bond—together they also represent alkanediyl (alkylene), where one hydrocarbon component of the alkanediyl grouping may be replaced by O (oxygen) and/or N (nitrogen) and/or an S(O)ₙ grouping and/or a (C=Q¹) grouping,
R⁷ represents in each case optionally substituted alkoxy, alkylthio, alkylamino, dialkylamino, alkenyloxy, alkenylthio, alkenylamino, alkynyloxy, alkynylthio, alkynylamino, cycloalkyloxy, cycloalkylthio, cycloalkylamino, cycloalkylalkoxy, cycloalkylalkylthio, cycloalkylalkylamino, arylalkoxy, arylalkylthio or arylalkylamino,
R⁸ represents in each case optionally substituted alkoxy, alkylamino, dialkylamino, alkenyloxy, alkenylthio, alkenylamino, alkynyloxy, alkynylthio, alkynylamino, cycloalkyloxy, cycloalkylthio, cycloalkylamino, cycloalkylalkoxy, cycloalkylalkylthio, cycloalkylalkylamino, arylalkoxy, arylalkylthio or arylalkylamino, or together with R⁷ represents in each case optionally substituted thiaalkyleneoxy, thiaalkyleneamino, thiaalkenyleneamino, alkylenediamino or alkenylenediamino in which optionally one methylene grouping is replaced by O (oxygen), S (sulfur) or N (nitrogen),
m represents the number 0 or 1,
n represents the number 0, 1 or 2,
A¹ represents a single bond or optionally substituted alkanediyl (alkylene),
A² represents optionally substituted alkanediyl (alkylene), and
Q³ represents O (oxygen) or S (sulfur).

The invention also provides products of the reaction of the compounds of the formula (I) with acidic and basic compounds (“acid adducts” and “salts”) suitable for use as crop protection agents and pesticides.

Depending on the nature and the number of substituents, the compounds of the formula (I) may, if appropriate, be present as geometrical and/or optical isomers or corresponding isomer mixtures of varying composition. The invention provides both the pure isomers and the isomer mixtures.

The formula (I) provides a general definition of the compounds according to the invention. In the definitions, the hydrocarbon chains, alkyl or alkenyl are in each case straight-chain or branched—including in combination with heteroatoms, such as in alkoxy. Optionally substituted radicals may be mono- or polysubstituted, where in the case of polysubstitution the substituents may be identical or different. Halogen is to be understood as meaning fluorine, chlorine, bromine and iodine.

Preferred substituents or ranges of the radicals present in the formulae given above and below are listed below.
R preferably represents alkyl having 1 to 6 carbon atoms which is mono- or polysubstituted by halogen.
R¹ preferably represents hydrogen, hydroxyl, in each case optionally hydroxyl-, cyano-, carboxyl-, carbamoyl-, halogen-, C₁-C₆-alkoxy- or C₁-C₆-alkoxycarbonyl-substituted alkyl, alkylcarbonyl, alkoxy, alkoxycarbonyl, alkylcarbonyloxy or alkoxycarbonyloxy having in each case 1 to 8 carbon atoms in the alkyl groups, in each case optionally halogen-substituted alkylsulfonyl or alkylsulfonyloxy having in each case 1 to 8 carbon atoms, in each case optionally cyano-, carboxyl-, carbamoyl-, halogen- or C₁-C₆-alkoxy-carbonyl-substituted alkenyl, alkynyl, alkenyloxy or alkynyloxy having in each case 2 to 8 carbon atoms, in each case optionally cyano-, carboxyl-, carbamoyl-, halogen-, C₁-C₄-alkyl-, C₁-C₄-haloalkyl- or C₁-C₄-alkoxy-carbonyl-substituted cycloalkyl, cycloalkyloxy, cycloalkylalkyl, cycloalkylalkoxy, cycloalkylcarbonyl, cycloalkylsulfonyl or cycloalkylsulfonyloxy having in each case 3 to 6 carbon atoms in the cycloalkyl group and, if appropriate, 1 to 6 carbon atoms in the alkyl moiety, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl, aryloxy, arylalkyl, arylalkoxy, aryl-carbonyl, arylsulfonyl or arylsulfonyloxy having in each case 6 or 10 carbon atoms in the aryl group and, if appropriate, 1 to 6 carbon atoms in the alkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted mono- or bicyclic heterocyclyl, heterocyclylalkyl, heterocyclylcarbonyl, heterocyclylsulfonyl or heterocyclylsulfonyloxy having in each case up to 10 carbon atoms and at least one heteroatom from the group consisting of N (nitrogen), O (oxygen) or an S(O)ₙ grouping and optionally additionally a (C=Q¹) grouping in the heterocyclyl group and, if appropriate, 1 to 6 carbon atoms in the alkyl moiety.
R³ preferably represents one of the groupings -A¹-R⁹ or -A¹-O—R⁶— or -A¹-S(O)ₙ—R⁶ or -A¹-N(R⁶)₂ or -A²-N(R⁶)—O—R⁶ or -A²-O—N(R⁶)₂ or -A²-O—N═C(R⁶)₂ or -A¹-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —N(R⁶)-A¹-(C=Q³)-R⁶ or -A¹-N(R⁶)—(C=Q³)-R⁶ or -A¹-C(R⁶)═N—O—R⁶ or -A¹-C(R⁶)═N(R⁶)—N(R⁶)₂ or -A²-C(R⁶)═N(R⁶)—N(R⁶)—(C=Q³)-(Q³)ₘR⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different.
R² and R³ together with the nitrogen atom to which both groups are attached preferably represent an optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted and optionally benzo-fused heterocycle which contains up to 8 carbon atoms and optionally one to three further nitrogen atoms and/or one oxygen atom and/or one S(O)ₙ grouping and optionally additionally one (C=Q¹) grouping.
R² and R³ preferably represent the grouping ═P(X)₃, where X represents amino, C₁-C₆-alkyl, C₁-C₆-alkylamino, C₃-C₆-cycloalkyl, C₃-C₆-cycloalkylamino, phenyl or phenylamino, where the radicals X may in each case be identical or different.
R⁴ preferably represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkyl, alkoxy, alkylamino or dialkylamino having in each case 1 to 8 carbon atoms in the alkyl groups, in each case optionally cyano-, carboxyl-, carbamoyl-, thiocarbamoyl-, halogen-, C₁-C₆-alkoxy-carbonyl-, C₁-C₆-alkylamino-carbonyl- or di-(C₁-C₄-alkyl)-amino-carbonyl-substituted alkenyl or alkynyl having in each case 2 to 8 carbon atoms, in each case optionally cyano-, carboxyl-, carbamoyl-, halogen-, C₁-C₄-alkyl- or C₁-C₄-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate, 1 to 6 carbon atoms in the alkyl moiety, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl, arylamino, N-alkyl-arylamino, arylalkyl, arylalkylamino, N-alkyl-arylalkylamino or N-cycloalkyl-arylalkylamino having in each case 6 or 10 carbon atoms in the aryl group and, if appropriate, 1 to 6 carbon atoms in the alkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted monocyclic or bicyclic heterocyclyl or heterocyclylalkyl having in each case up to 10 carbon atoms and at least one heteroatom from the group consisting of N (nitrogen), O (oxygen) or an S(O)ₙ grouping and optionally additionally a (C=Q¹) grouping in the heterocyclyl group and, if appropriate, 1 to 6 carbon atoms in the alkyl moiety.
R⁵ preferably represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkyl, alkoxy, alkylamino or dialkylamino having in each case 1 to 8 carbon atoms in the alkyl groups, in each case optionally cyano-, carboxyl-, carbamoyl-, thiocarbamoyl-, halogen-, C₁-C₆-alkoxy-carbonyl-, C₁-C₆-alkylamino-carbonyl- or di-(C₁-C₄-alkyl)-amino-carbonyl-substituted alkenyl or alkynyl having in each case 2 to 8 carbon atoms, in each case optionally cyano-, carboxyl-, carbamoyl-, halogen-, C₁-C₄-alkyl- or C₁-C₄-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate 1 to 6 carbon atoms in the alkyl moiety, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl, arylamino, N-alkyl-arylamino, arylalkyl, arylalkylamino, N-alkyl-arylalkylamino, N-cycloalkyl-arylalkylamino having in each case 6 or 10 carbon atoms in the aryl group, if appropriate 1 to 6 carbon atoms in the alkyl moiety and if appropriate 3 to 6 carbon atoms in the cycloalkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted monocyclic or bicyclic heterocyclyl or heterocyclylalkyl having in each case up to 10 carbon atoms and at least one heteroatom from the group consisting of N (nitrogen), O (oxygen) or an S(O)ₙ grouping in the heterocyclyl group and, if appropriate 1 to 6 carbon atoms in the alkyl moiety, or together with R⁴ represents in each case optionally cyano-, halogen- or C₁-C₆-alkyl-substituted and/or benzo-fused alkanediyl (alkylene), alkyleneamino or alkylenediamino, each of which has 2 to 6 carbon atoms in the alkylene grouping and is optionally interrupted by O (oxygen) and/or N (nitrogen) and/or an S(O)ₙ grouping and/or a (C=Q¹) grouping.
R⁶ preferably represents hydrogen, hydroxyl, amino, cyano, optionally cyano-, carboxyl-, carbamoyl-, thiocarbamoyl-, hydroxyl-, amino-, halogen-, C₁-C₆-alkoxy-, C₁-C₆-alkylthio-, C₁-C₆-alkylsulfinyl-, C₁-C₆-alkylsulfonyl-, C₁-C₆-alkylamino-, di-(C₁-C₄-alkyl)amino-, C₁-C₆-alkoxy-carbonyl-, C₁-C₆-alkylamino-carbonyl- or di-(C₁-C₄-alkyl)-amino-carbonyl-substituted alkyl having 1 to 8 carbon atoms, in each case optionally cyano-, carboxyl-, carbamoyl-, thiocarbamoyl-, halogen-, C₁-C₆-alkoxy-carbonyl-, C₁-C₆-alkylamino-carbonyl- or di-(C₁-C₄-alkyl)-amino-carbonyl-substituted alkenyl or alkynyl having in each case 2 to 8 carbon atoms, in each case optionally cyano-, carboxyl-, carbamoyl-, halogen-, C₁-C₄-alkyl-, C₁-C₄-haloalkyl- or C₁-C₄-alkoxy-carbonyl-substituted cycloalkyl, cycloalkenyl, cycloalkylalkyl or cycloalkenylalkyl having in each case 3 to 6 carbon atoms in the cycloalkyl or cycloalkenyl groups, and, if appropriate, 1 to 6 carbon atoms in the alkyl moiety, where the cycloalkyl groups each optionally contain a (C=Q¹) grouping, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl or arylalkyl having in each case 6 or 10 carbon atoms in the aryl groups and, if appropriate, 1 to 6 carbon atoms in the alkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted monocyclic or bicyclic heterocyclyl or heterocyclylalkyl having in each case up to 10 carbon atoms and at least one hetero building block from the group consisting of N (nitrogen), O (oxygen), S(O)ₙ and optionally additionally a (C=Q³) grouping in the heterocyclyl group and, if appropriate, 1 to 6 carbon atoms in the alkyl moiety, or, if two groupings R⁶ are located in geminal position on a C═C double bond, these groupings together optionally also represent alkanediyl having 3 to 6 carbon atoms, where one (hydro)carbon unit (C, CH, CH₂) Of the alkanediyl grouping may be replaced by O (oxygen), N (nitrogen), an S(O)ₙ grouping or a (C=Q¹) grouping.
R⁷ preferably represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkoxy, alkylthio, alkylamino or dialkylamino having in each case 1 to 8 carbon atoms in the alkyl groups, represents in each case optionally cyano- or halogen-substituted alkenyloxy, alkenylthio, alkenylamino, alkynyloxy, alkynylthio or alkynylamino having in each case 3 to 8 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 4 carbon atoms in the alkyl moiety, or represents in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted arylalkoxy, arylalkylthio or arylalkylamino having in each case 6 or 10 carbon atoms in the aryl group and 1 to 4 carbon atoms in the alkyl moiety.
R⁸ preferably represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkoxy, alkylamino or dialkylamino having in each case 1 to 8 carbon atoms in the alkyl groups, represents in each case optionally cyano- or halogen-substituted alkenyloxy, alkenylthio, alkenylamino, alkynyloxy, alkynylthio or alkynylamino having in each case 3 to 8 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 4 carbon atoms in the alkyl moiety, or represents in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted arylalkoxy, arylalkylthio or arylalkylamino having in each case 6 or 10 carbon atoms in the aryl group and 1 to 4 carbon atoms in the alkyl moiety, or together with R⁷ represents in each case optionally cyano-, hydroxy-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted thiaalkyleneoxy, thiaalkyleneamino, thiaalkenyleneamino, alkylenediamino or alkenylenediamino having in each case up to 5 carbon atoms, where in each case one methylene grouping is optionally replaced by O (oxygen), S (sulfur) or N (nitrogen).
A¹ preferably represents a single bond or represents optionally hydroxyl-, cyano-, carboxyl-, phenyl-, C₁-C₆-alkyl-, C₁-C₆-haloalkyl-, C₁-C₆-alkoxy-, C₁-C₆-haloalkoxy- or C₁-C₆-alkoxy-carbonyl-substituted alkanediyl (alkylene) having 1 to 6 carbon atoms.
A² preferably represents optionally hydroxyl-, cyano-, carboxyl-, C₁-C₆-alkyl-, C₁-C₆-haloalkyl-, C₁-C₆-alkoxy-, C₁-C₆-haloalkoxy- or C₁-C₆-alkoxy-carbonyl-substituted alkanediyl (alkylene) having 1 to 6 carbon atoms.
R particularly preferably represents alkyl which has 1 to 4 carbon atoms and is mono- or polysubstituted by fluorine, chlorine and/or bromine.
Q¹ particularly preferably represents oxygen.
Q² particularly preferably represents the grouping

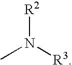

R¹ particularly preferably represents hydrogen, hydroxyl, in each case optionally cyano-, carboxyl-, carbamoyl-, halogen-, C₁-C₄-alkoxy- or C₁-C₄-alkoxy-carbonyl-substituted alkyl, alkylcarbonyl, alkoxy, alkoxycarbonyl, alkylcarbonyloxy or alkoxycarbonyloxy having in each case 1 to 6 carbon atoms in the alkyl groups, in each case optionally halogen-substituted alkylsulfonyl or alkylsulfonyloxy having in each case 1 to 6 carbon atoms, in each case optionally cyano-, carboxyl-, carbamoyl-, halogen- or C₁-C₄-alkoxy-carbonyl-substituted alkenyl, alkynyl, alkenyloxy or alkynyloxy having in each case 3 to 6 carbon atoms, in each case optionally cyano-, carboxyl-, carbamoyl-, halogen-, C₁-C₃-alkyl- or C₁-C₃-haloalkyl-substituted cycloalkyl, cycloalkyloxy, cycloalkylalkyl, cycloalkylalkoxy, cycloalkylcarbonyl, cycloalkylsulfonyl or cycloalkylsulfonyloxy having in each case 3 to 6 carbon atoms in the cycloalkyl group and, if appropriate, 1 to 4 carbon atoms in the alkyl moiety, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted aryl, aryloxy, arylalkyl, arylalkoxy, arylcarbonyl, arylsulfonyl or arylsulfonyloxy having in each case 6 or 10 carbon atoms in the aryl group and, if appropriate, 1 to 4 carbon atoms in the alkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted heterocyclyl, heterocyclylalkyl, heterocyclylcarbonyl, heterocyclylsulfonyl or heterocyclylsulfonyloxy having in each case up to 8 carbon atoms and at least one heteroatom from the group consisting of N (nitrogen), O (oxygen) or an S(O)ₙ grouping and optionally additionally a (C=Q¹) grouping in the heterocyclyl group and, if appropriate, 1 to 4 carbon atoms in the alkyl moiety.
R² particularly preferably represents one of the groupings -A¹-R⁶ or -A²-O—R⁶— or -A¹-S(O)ₙ—R⁶ or -A²-N(R⁶)₂ or -A²-N(R⁶)—O—R⁶ or -A²-O—N(R⁶)₂ or -A²-O—N═C(R⁶)₂ or -A²-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A²-(C=Q³)-N(R⁶)₂ or -A²-(C=Q³)-N(R⁶)OR⁶ or -A²-O—(C=Q³)-R⁶ or -A²-N(R⁶)—(C=Q³)-R⁶, where the radicals R⁶—if a plurality of them is present in the same groupings—may be identical or different.
R³ particularly preferably represents one of the groupings -A¹-R⁶ or -A¹-O—R⁶— or -A¹-S(O)ₙ—R⁶ or -A¹-N(R⁶)₂ or -A²-N(R⁶)—O—R⁶ or -A²-O—N(R⁶)₂ or -A²-O—N═C(R⁶)₂ or -A¹-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —N(R⁶)-A¹-(C=Q³)-R⁶ or -A¹-N(R⁶)—(C=Q³)-R⁶ or -A¹-C(R⁶)═N—O—R⁶ or -A¹-C(R⁶)═N(R⁶)—N(R⁶)₂ or -A²-C(R⁶)N(R⁶)—N(R⁶)—(C=Q³)-(Q³)ₘ-R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different.
R² and R³ together with the nitrogen atom which is attached to the two groups particularly preferably represent an optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted monocyclic or bicyclic heterocycle which contains up to 8 carbon atoms and optionally one to three further nitrogen atoms and/or one oxygen atom and/or one (SO)ₙ grouping and optionally additionally a (C=Q¹) grouping or.
R² and R³ together particularly preferably represent the grouping ═P(X)₃, where X represents amino, C₁-C₄-alkyl, C₁-C₄-alkylamino, C₃-C₆-cycloalkyl, C₃-C₆-cycloalkylamino, phenyl or phenylamino and where the radicals X may in each case be identical or different.
R⁴ particularly preferably represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkyl, alkoxy, alkylamino or dialkylamino having in each case 1 to 6 carbon atoms in the alkyl groups, in each case optionally cyano-, carboxyl-, carbamoyl-, thiocarbamoyl-, halogen-, C₁-C₄-alkoxy-carbonyl-, C₁-C₄-alkylamino-carbonyl- or di-(C₁-C₃-alkyl)-amino-carbonyl-substituted alkenyl or alkynyl having in each case 2 to 6 carbon atoms, in each case optionally cyano-, carboxyl-, carbamoyl-, halogen-, C₁-C₃-alkyl- or C₁-C₃-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate, 1 to 4 carbon atoms in the alkyl moiety, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl, di-(C₁-C₃-alkyl)-amino-carbonyl- or di-(C₁-C₃-alkyl)-amino-sulfonyl-substituted aryl, arylamino, N-alkyl-arylamino, arylalkyl, arylalkylamino, N-alkyl-arylalkylamino or N-cycloalkyl-arylalkylamino having in each case 6 or 10 carbon atoms in the aryl group and, if appropriate, 1 to 4 carbon atoms in the alkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted heterocyclyl or heterocyclylalkyl having in each case up to 8 carbon atoms and at least one heteroatom from the group consisting of N (nitrogen), O (oxygen) or an S(O)ₙ grouping and optionally additionally a (C═N—R¹) grouping in the heterocyclyl group and, if appropriate, 1 to 4 carbon atoms in the alkyl moiety.
R⁵ particularly preferably represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)amino-, C₁-C₄-alkoxy-carbonyl-, C₁-C₄-alkylamino-carbonyl- or di-(C₁-C₃-alkyl)-amino-carbonyl-substituted alkyl, alkoxy, alkylamino or dialkylamino having in each case 1 to 6 carbon atoms in the alkyl groups, in each case optionally cyano-, carboxyl-, carbamoyl-, thiocarbamoyl-, halogen-, C₁-C₄-alkoxy-carbonyl-, C₁-C₄-alkylamino-carbonyl- or di-(C₁-C₃-alkyl)-amino-carbonyl-substituted alkenyl or alkynyl having in each case 2 to 6 carbon atoms, in each case optionally cyano-, carboxyl-, carbamoyl-, halogen-, C₁-C₃-alkyl- or C₁-C₃-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate, 1 to 4 carbon atoms in the alkyl moiety, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl, di-(C₁-C₃-alkyl)-amino-carbonyl- or di-(C₁-C₃-alkyl)-amino-sulfonyl-substituted aryl, arylamino, N-alkyl-arylamino, arylalkyl, arylalkylamino, N-alkyl-arylalkylamino or N-cycloalkyl-arylalkylamino having in each case 6 or 10 carbon atoms in the aryl group, if appropriate 1 to 4 carbon atoms in the alkyl moiety and if appropriate 3 to 6 carbon atoms in the cycloalkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted heterocyclyl or heterocyclylalkyl having in each case up to 8 carbon atoms and at least one heteroatom from the group consisting of N (nitrogen), O (oxygen) or an S(O)ₙ grouping in the heterocyclyl group and, if appropriate, 1 to 4 carbon atoms in the alkyl moiety, or together with R⁴ represents in each case optionally cyano-, halogen- or C₁-C₄-alkyl-substituted and/or benzo-fused alkanediyl (alkylene), alkyleneamino or alkylenediamino, each of which has 2 to 5 carbon atoms in the alkylene grouping and is optionally interrupted by 0 (oxygen) and/or N (nitrogen) and/or an S(O)ₙ grouping and/or a (C=Q¹) grouping.
R⁶ particularly preferably represents hydrogen, in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkyl, alkoxy, alkylamino, alkoxyamino, dialkylamino or N-alkyl-alkoxyamino having in each case 1 to 6 carbon atoms in the alkyl groups, in each case optionally cyano-, carboxyl-, carbamoyl-, thiocarbamoyl-, halogen-, C₁-C₄-alkoxy-carbonyl-, C₁-C₄-alkylamino-carbonyl- or di-(C₁-C₃-alkyl)-amino-carbonyl-substituted alkenyl, alkenyloxy, alkenylamino, N—C₁-C₄-alkyl-alkenyl-amino, alkynyl, alkynyloxy, alkynylamino or N—C₁-C₄-alkyl-alkynylamino having in each case 2 to 6 carbon atoms in the alkenyl or alkynyl groups, in each case optionally cyano-, carboxyl-, carbamoyl-, halogen-, C₁-C₃-alkyl- or C₁-C₃-haloalkyl-substituted cycloalkyl, cycloalkyloxy, cycloalkylamino, N-alkyl-cycloalkylamino, cycloalkylalkyl, cycloalkylalkoxy, cycloalkylalkylamino or N-alkyl-cycloalkylalkylamino having in each case 3 to 6 carbon atoms in the cycloalkyl groups and, if appropriate, 1 to 4 carbon atoms in the alkyl moiety, where the cycloalkyl groups each optionally contain a (C=Q¹) grouping, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted aryl, aryloxy, arylthio, arylamino, N-alkyl-arylamino, arylalkyl, arylalkoxy, arylalkylthio, arylalkylamino or N-alkyl-arylalkylamino having in each case 6 or 10 carbon atoms in the aryl groups and, if appropriate, 1 to 4 carbon atoms in the alkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted monocyclic or bicyclic heterocyclyl or heterocyclylalkyl having in each case up to 8 carbon atoms and at least one hetero building block from the group consisting of N (nitrogen), O (oxygen), S(O)ₙ and optionally additionally a (C=Q³) grouping in the heterocyclyl group and, if appropriate, 1 to 4 carbon atoms in the alkyl moiety, or, if two groupings R⁶ are located in geminal position on a C═C double bond, these groupings together optionally also represent alkanediyl (divalent hydrocarbon chain, “alkylene”) having 4 or 5 carbon atoms, where one (hydro)carbon unit (C, CH, CH₂) may be replaced by O (oxygen), N (nitrogen), an S(O)ₙ grouping or a (C=Q¹) grouping.
R⁷ particularly preferably represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkoxy, alkylthio, alkylamino or dialkylamino having in each case 1 to 6 carbon atoms in the alkyl groups, represents in each case optionally cyano- or halogen-substituted alkenyloxy, alkenylthio, alkenylamino, alkynyloxy, alkynylthio or alkynylamino having in each case 3 to 6 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 3 carbon atoms in the alkyl moiety, or represents in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted phenylalkoxy, phenylalkylthio or phenylalkylamino having 1 to 3 carbon atoms in the alkyl moiety.
R⁸ particularly preferably represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkoxy, alkylamino or dialkylamino having in each case 1 to 6 carbon atoms in the alkyl groups, represents in each case optionally cyano- or halogen-substituted alkenyloxy, alkenylthio, alkenylamino, alkynyloxy, alkynylthio or alkynylamino having in each case 3 to 6 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 3 carbon atoms in the alkyl moiety, or represents in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted phenylalkoxy, phenylalkylthio or phenyl-alkylamino having 1 to 3 carbon atoms in the alkyl moiety, or together with R⁷ represents in each case optionally cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted thiaalkyleneoxy, thiaalkyleneamino, thiaalkenyleneamino, alkylenediamino or alkenylenediamino having in each case up to 4 carbon atoms, where in each case one (hydro)carbon unit (C, CH, CH₂) is optionally replaced by O (oxygen), S (sulfur) or N (nitrogen).
A¹ particularly preferably represents a single bond or represents optionally hydroxyl-, cyano-, carboxyl-, phenyl-, C₁-C₄-alkyl-, C₁-C₄-haloalkyl-, C₁-C₄-alkoxy-, C₁-C₄-haloalkoxy- or C₁-C₄-alkoxy-carbonyl-substituted alkanediyl (alkylene) having 1 to 4 carbon atoms.
A² particularly preferably represents optionally hydroxyl-, cyano-, carboxyl-, C₁-C₄-alkyl-, C₁-C₄-haloalkyl-, C₁-C₄-alkoxy-, C₁-C₄-haloalkoxy- or C₁-C₄-alkoxy-carbonyl-substituted alkanediyl (alkylene) having 1 to 4 carbon atoms.
R very particularly preferably represents methyl, ethyl, n- or i-propyl, each of which is mono- or polysubstituted by fluorine and/or chlorine.
R¹ very particularly preferably represents hydrogen, hydroxyl, in each case optionally cyano-, carboxyl-, carbamoyl-, fluorine-, chlorine-, methoxy-, ethoxy-, n- or i-propoxy-, methoxycarbonyl-, ethoxycarbonyl-, n- or i-propoxycarbonyl-substituted methyl, ethyl, n- or i-propyl, n-, i-, s- or t-butyl, acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, acetyloxy, propionyloxy, n- or i-butyroyloxy, methoxycarbonyloxy, ethoxycarbonyloxy, n- or i-propoxycarbonyloxy, in each case optionally fluorine-, chlorine- and/or bromine-substituted methylsulfonyl, ethylsulfonyl, n- or i-propylsulfonyl, n-, i-, s- or t-butylsulfonyl, methylsulfonyloxy, ethylsulfonyloxy, n- or i-propyl-sulfonyloxy, in each case optionally cyano-, fluorine-, chlorine-, bromine-, methoxycarbonyl-, ethoxycarbonyl-, n- or i-propoxycarbonyl-substituted propenyl, butenyl, propynyl, butynyl, propenyloxy, butenyloxy, propynyloxy or butynyloxy, in each case optionally cyano-, fluorine-, chlorine-, bromine-, methyl-, ethyl-, n- or i-propyl- or trifluoromethyl-substituted cyclopropyl, cyclobutyl, cyclopentyl, cyclohexyl, cyclopentyloxy, cyclohexyloxy, cyclopropylmethyl, cyclopentylmethyl, cyclohexyl-methyl, cyclopentylmethoxy, cyclohexylmethoxy, cyclopropylcarbonyl, cyclobutyl-carbonyl, cyclopentylcarbonyl, cyclopropylsulfonyl, cyclobutylsulfonyl, cyclopentyl-sulfonyl, cyclohexylsulfonyl, cyclopentylsulfonyloxy or cyclohexylsulfonyloxy, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, n- or i-propylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylamino-carbonyl- or dimethylaminosulfonyl-substituted phenyl, naphthyl, phenoxy, naphthyl-oxy, benzyl, phenylethyl, phenylmethoxy, phenylethoxy, benzoyl, naphthoyl, phenylsulfonyl, naphthylsulfonyl, phenylsulfonyloxy or naphthylsulfonyloxy, or in each case optionally nitro-, cyano-, hydroxyl-, carboxyl-, carbamoyl, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, n- or i-propylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted heterocyclyl, heterocyclylmethyl, heterocyclylsulfonyl or heterocyclylsulfonyloxy, where the heterocyclyl grouping is in each case selected from the group consisting of furyl, thienyl, pyrazolyl, pyridyl, pyrimidyl.
R² very particularly preferably represents one of the groupings -A¹-R⁶ or -A²-O—R⁶— or -A¹-S(O)ₙ—R⁶ or -A²-N(R⁶)₂ or -A²-N(R⁶)—O—R⁶ or -A²-O—N(R⁶)₂ or -A²-O—N═C(R⁶)₂ or -A²-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A²-O—(C=Q³)-R⁶ or -A²-NH—(C=Q³)-R⁶, where the radical R⁶— if a plurality of them is present in the same groupings—may be identical or different.
R³ very particularly preferably represents one of the groupings -A¹-R⁶ or -A¹-O—R⁶— or -A¹-S(O)ₙ—R⁶ or -A¹-N(R⁶)₂ or -A²-N(R⁶)—O—R⁶ or -A²-O—N(R⁶)₂ or -A²-O—N═C(R⁶)₂ or -A¹-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —NH-A¹-(C=Q³)-R⁶ or -A¹-NH—(C=Q³)-R⁶ or -A¹-C(R⁶)═N—O—R⁶ or -A¹-C(R⁶)═N(R⁶)—N(R⁶)₂ or -A²-C(R⁶)═N(R⁶)—N(R⁶)—(C=Q³)-(Q³)ₘ-R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different.
R² and R³ very particularly preferably represent the grouping ═CR⁷R⁸.
R² and R³ together with the nitrogen atom to which the two groups are attached very particularly preferably represent an optionally cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, trifluoromethoxy-, methylthio-, ethylthio-, n- or i-propylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted and optionally benzo-fused heterocycle from the group consisting of pyrrolidinyl, oxopyrrolidinyl, piperidinyl, oxopiperidinyl, morpholinyl, oxomorpholinyl.
R⁴ very particularly preferably represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propyl-amino-carbonyl-, dimethylaminocarbonyl- or diethylaminocarbonyl-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino, n-, i-, s- or t-butylamino, dimethylamino or diethylamino, in each case optionally cyano-, fluorine-, chlorine-, bromine-, methoxycarbonyl-, ethoxycarbonyl-, n- or i-propoxycarbonyl-, methylamino-carbonyl-, ethylaminocarbonyl-, n- or i-propylamino-carbonyl-, dimethylamino-carbonyl- or diethylaminocarbonyl-substituted ethenyl, propenyl, butenyl, ethynyl, propynyl or butynyl, in each case optionally cyano-, fluorine-, chlorine-, bromine-, methyl-, ethyl- or trifluoromethyl-substituted cyclopropyl, cyclobutyl, cyclopentyl, cyclohexyl, cyclopropylamino, cyclobutylamino, cyclopentylamino, cyclohexylamino, N-methyl-cyclopropylamino, N-methyl-cyclobutylamino, N-methyl-cyclopentylamino, N-methyl-cyclohexylamino, cyclopentenyl, cyclohexenyl, cyclopropylmethyl, cyclobutylmethyl, cyclopentylmethyl or cyclohexylmethyl, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenyl, naphthyl, phenylamino, naphthylamino, N-methyl-phenylamino, N-ethyl-phenylamino, benzyl, phenylethyl, phenylmethylamino, phenylethyl-amino, N-methyl-phenylmethylamino, N-ethyl-phenylmethylamino or N-cyclopropyl-phenylmethylamino, or in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxy-carbonyl-, dimethylaminocarbonyl- or dimethylaminosulfonyl-substituted heterocyclyl or heterocyclylmethyl, where heterocyclyl is selected from the group consisting of furyl, tetrahydrofuryl, thienyl, pyrazolyl, pyrazolinyl, oxazolyl, oxazolinyl, isoxazolyl, thiazolyl, pyridyl, pyrimidyl.
R⁵ very particularly preferably represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propyl-amino-carbonyl-, dimethylaminocarbonyl- or diethylaminocarbonyl-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino, n-, i-, s- or t-butylamino, dimethylamino or diethylamino, in each case optionally cyano-, fluorine-, chlorine-, bromine-, methoxycarbonyl-, ethoxycarbonyl-, n- or i-propoxycarbonyl-, methylamino-carbonyl-, ethylaminocarbonyl-, n- or i-propylamino-carbonyl-, dimethylamino-carbonyl- or diethylaminocarbonyl-substituted ethenyl, propenyl, butenyl, ethynyl, propynyl or butynyl, in each case optionally cyano-, fluorine-, chlorine-, bromine-, methyl-, ethyl- or trifluoromethyl-substituted cyclopropyl, cyclobutyl, cyclopentyl, cyclohexyl, cyclopropylamino, cyclobutylamino, cyclopentylamino, cyclohexylamino, cyclopentenyl, cyclohexenyl, cyclopropylmethyl, cyclobutylmethyl, cyclopentylmethyl or cyclohexylmethyl, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenyl, naphthyl, phenylamino, naphthylamino, N-methyl-phenylamino, N-ethyl-phenylamino, benzyl, phenylethyl, phenylmethylamino, phenylethylamino, N-methyl-phenylmethylamino, N-ethyl-phenylmethylamino or N-cyclopropyl-phenylmethylamino, or in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted heterocyclyl or heterocyclylmethyl, where heterocyclyl is selected from the group consisting of furyl, tetrahydrofuryl, thienyl, pyrazolyl, pyrazolinyl, oxazolyl, oxazolinyl, isoxazolyl, thiazolyl, pyridyl, pyrimidyl, or together with R⁴ represents in each case optionally cyano-, fluorine-, chlorine-, bromine-, methyl-, ethyl-, n- or i-propyl-substituted and/or benzo-fused propane-1,3-diyl, butane-1,4-diyl, pentane-1,5-diyl, 1-azapropane-1,3-diyl, 1-azabutane-1,4-diyl, 1-azapentane-1,5-diyl, 1,3-diazapropane-1,3-diyl, 1,4-diazabutane-1,4-diyl or 1,5-diazapentane-1,5-diyl, each of which is optionally interrupted by O, S, N, (C═O), (C═S), (SO) or (SO₂).
R⁶ very particularly preferably represents hydrogen, in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylamino-carbonyl-, n- or i-propylaminocarbonyl-, dimethylaminocarbonyl- or diethylamino-carbonyl-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino, n-, i-, s- or t-butylamino, methoxyamino, ethoxyamino, n- or i-propoxyamino, n-, i-, s- or t-butoxyamino, dimethylamino, diethylamino, N-methyl-methoxyamino, N-ethyl-methoxyamino or N-methyl-ethoxyamino, in each case optionally cyano-, fluorine-, chlorine-, bromine-, methoxycarbonyl-, ethoxycarbonyl-, n- or i-propoxycarbonyl-, methylaminocarbonyl-, ethylaminocarbonyl-, n- or i-propylaminocarbonyl-, dimethylaminocarbonyl- or diethylaminocarbonyl-substituted ethenyl, propenyl, butenyl, propenyloxy, butenyloxy, propenylamino, butenylamino, N-methyl-propenylamino, N-ethyl-propenylamino, N-methyl-butenylamino, N-ethyl-butenylamino, ethynyl, propynyl, butynyl, propynyloxy, butynyloxy, propynylamino, butynylamino, N-methyl-propynylamino, N-ethyl-propynylamino, N-methyl-butynylamino or N-ethyl-butynyl-amino, in each case optionally cyano-, fluorine-, chlorine-, bromine-, methyl-, ethyl- or trifluoromethyl-substituted cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, N-methyl-cyclopropylamino, N-methyl-cyclobutylamino, N-methyl-cyclopentylamino, N-methyl-cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethylamino, cyclobutylmethylamino, cyclopentylmethylamino, cyclohexylmethylamino, N-methyl-cyclopropylmethylamino, N-methyl-cyclobutylmethylamino, N-methyl-cyclopentylmethylamino or N-methyl-cyclohexylmethylamino, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxy-carbonyl-, dimethylaminocarbonyl- or dimethylaminosulfonyl-substituted phenyl, naphthyl, phenoxy, naphthyloxy, phenylthio, naphthylthio, phenylamino, naphthylamino, N-methyl-phenylamino, benzyl, phenylmethyl, phenylethyl, phenylmethoxy, phenylethoxy, phenylmethylthio, phenylethylthio, phenylmethylamino, phenylethylamino or N-methyl-phenylmethyl-mino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted heterocyclyl or heterocyclylmethyl, where the heterocyclyl grouping is selected from the group consisting of oxetanyl, furyl, tetrahydrofuryl, oxotetrahydrofuryl, thienyl, tetrahydrothienyl, oxo- and dioxo-tetrahydrothienyl, pyrrolyl, pyrrolinyl, pyrrolidinyl, oxopyrrolidinyl, dioxo-pyrrolidinyl, pyrazolyl, pyrazolinyl, imidazolyl, imidazolinyl, oxazolyl, oxazolinyl, thiazolyl, thiazolinyl, oxadiazolyl, thiadiazolyl, tetrahydropyranyl, oxotetrahydro-pyranyl, benzotetrahydropyranyl, tetrahydrothiopyranyl, oxo- and dioxotetrahydrothio-pyranyl, dioxacyclohexyl, piperidinyl, oxopiperidinyl, morpholinyl, oxomorpholinyl, pyridinyl, pyrimidinyl.
R⁷ very particularly preferably represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propylaminocarbonyl-, dimethylaminocarbonyl- or diethylaminocarbonyl-substituted methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino or dipropylamino, represents in each case optionally cyano-, fluorine-, chlorine- or bromine-substituted propenyloxy, butenyloxy, pentenyloxy, propenylthio, butenylthio, pentenylthio, propenylamino, butenylamino, pentenylamino, propynyloxy, butynyloxy, pentynyloxy, propynylthio, butynylthio, pentynylthio, propynylamino, butynylamino or pentynylamino, represents in each case optionally cyano-, fluorine-, chlorine-, bromine-, methyl-, ethyl-, n- or i-propyl- or trifluoromethyl-substituted cyclopentyloxy, cyclohexyloxy, cyclopentylthio, cyclohexylthio, cyclopentylamino, cyclohexylamino, cyclopentylmethoxy, cyclohexylmethoxy, cyclopentylmethylthio, cyclohexylmethylthio, cyclopentylmethylamino or cyclohexylmethylamino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenylmethoxy, phenylmethylthio or phenylmethylamino.
R⁸ very particularly preferably represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propylaminocarbonyl-, dimethylaminocarbonyl- or diethylaminocarbonyl-substituted methoxy, ethoxy, n- or i-propoxy, n-, i-, s- or t-butoxy, methylamino, ethylamino, n- or i-propylamino, n-, i-, s- or t-butylamino, dimethylamino, diethylamino or dipropyl-amino, represents in each case optionally cyano-, fluorine-, chlorine- or bromine-substituted propenyloxy, butenyloxy, pentenyloxy, propenylthio, butenylthio, pentenyl-thio, propenylamino, butenylamino, pentenylamino, propynyloxy, butynyloxy, pentynyloxy, propynylthio, butynylthio, pentynylthio, propynylamino, butynylamino or pentynylamino, represents in each case optionally cyano-, fluorine-, chlorine-, bromine-, methyl-, ethyl-, n- or i-propyl- or trifluoromethyl-substituted cyclopentyloxy, cyclohexyloxy, cyclopentylthio, cyclohexylthio, cyclopentylamino, cyclohexylamino, cyclopentylmethoxy, cyclohexylmethoxy, cyclopentylmethylthio, cyclohexylmethylthio, cyclopentylmethylamino or cyclohexylmethylamino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenylmethoxy, phenylmethylthio or phenylmethylamino, or together with R⁷ represents in each case optionally cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, methylaminocarbonyl-, ethylaminocarbonyl-, n- or i-propylamino-carbonyl-, dimethylamino-carbonyl- or dimethylaminosulfonyl-substituted thiaethyleneoxy, thiapropylene-oxy, thiaethyleneamino, thiapropyleneamino, ethylenediamino or propylendiamino.
A¹ very particularly preferably represents a single bond, represents methylene, or represents in each case optionally hydroxyl-, cyano-, carboxyl-, methyl-, ethyl-, n- or i-propyl-, trifluoromethyl-, methoxy-, ethoxy-, n- or i-propoxy-, trifluoromethoxy-, methoxycarbonyl-, ethoxycarbonyl-, n- or i-propoxycarbonyl-substituted ethane-1,1-diyl, ethane-1,2-diyl, propane-1,1-diyl, propane-1,2-diyl or propane-1,3-diyl.
A² very particularly preferably represents methylene or represents in each case optionally hydroxyl-, cyano-, carboxyl-, methyl-, ethyl-, n- or i-propyl-, trifluoromethyl-, methoxy-, ethoxy-, n- or i-propoxy-, trifluoromethoxy-, methoxycarbonyl-, ethoxycarbonyl-, n- or i-propoxycarbonyl-substituted ethane-1,1-diyl, ethane-1,2-diyl, propane-1,1-diyl, propane-1,2-diyl or propane-1,3-diyl.
R most preferably represents CF₃.

Particularly preferred groups of compounds of the formula (I) are defined below.

Group 1: Compounds of the formula (I) in which R represents CF₃, Q¹ represents oxygen, R² represents hydrogen and Q² represents the grouping

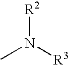

and the remaining substituents have one of the meanings mentioned above.

Group 2: Compounds of the formula (I) in which R represents CF₃, Q¹ represents oxygen, R³ represents CH₂CN and Q² represents the grouping

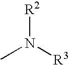

and the remaining substituents have one of the meanings mentioned above.

Group 3: Compounds of the formula (I) in which R represents CF₃ Q¹ represents oxygen and Q² represents the grouping

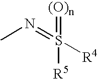

and the remaining substituents have one of the meanings mentioned above.

Preference according to the invention is given to the compounds of the formula (I) which contain a combination of the meanings listed above as being preferred.

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

The general or preferred radical definitions listed above apply both to the end products of the formula (I) and, correspondingly, to the starting materials or intermediates required in each case for the preparation. These radical definitions can be combined with one another as desired, i.e. including combinations between the given preferred ranges. It is also possible for individual definitions not to apply.

Furthermore, it has been found that the substituted pyridazinecarboxamides and derivatives thereof of the formula (I) are obtained when—process (a)—

Pyridazinecarboxylic acids of the formula (II),

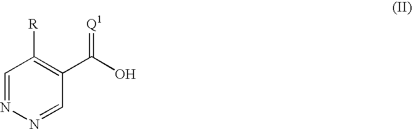

in which R and Q¹ are as defined above, or reactive derivatives thereof, are reacted with nitrogen compounds of the general formula (III)

H-Q² (III)

in which Q² is as defined above, if appropriate in the presence of one or more reaction auxiliaries and if appropriate in the presence of one or more diluents and, if appropriate, the resulting compounds of the formula (I) are subjected to further reactions according to customary methods to convert them into other compounds of the formula (I) according to the above definitions.

Routes for converting compounds of the formula (I) into other compounds of the formula (I) are illustrated below:

Conversion of monosubstituted pyridazinecarboxamides into disubstituted pyridazine-carboxamides—process (b)—

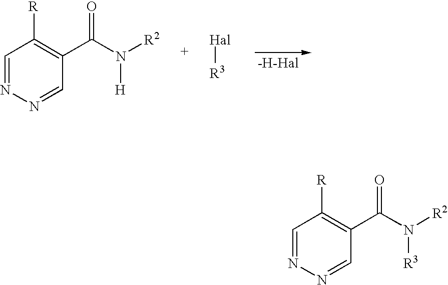

in which R¹, R², R³ are as defined above and Hal represents a leaving group, preferably halogen.

Reaction of pyridazinecarboxamides with chlorinating agents (for example chlorine or t-butyl hypochlorite), isolation of the resulting N-chloroamides (X═H) or N,N-dichloroamides (X═Cl) and their reaction with sulfides (cf. WO 01/070692)—process (c)—

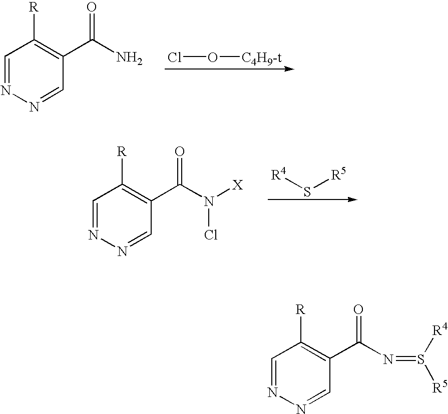

Reaction of sulfenylated pyridazinecarboxamides with alkylating agents (cf. WO 03/028458)—process (d)—

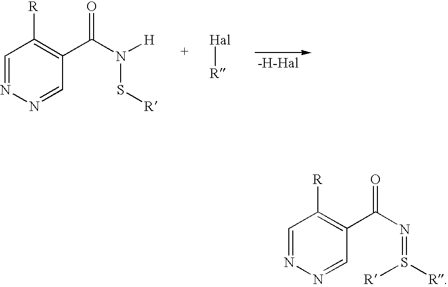

Using, for example, 5-trifluoromethylpyridazine-4-carbonyl chloride and methylamine as starting materials, the course of reaction in the process (a) according to the invention can be illustrated by the formula scheme below:

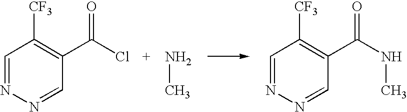

The formula (II) provides a general definition of the substituted pyridazinecarboxamides to be used as starting materials in the process (a) according to the invention for preparing the compounds of the general formula (I). In the general formula (II), Q¹ and R preferably or in particular have those meanings which have already been mentioned above, in connection with the description of the compounds of the general formula (I) according to the invention, as being preferred or as being particularly preferred for Q¹ and R.

Reactive derivatives of the pyridazinecarboxylic acids of the formula (I) which may be mentioned are the corresponding carbonyl halides, in particular the corresponding carbonyl chlorides, and also corresponding carboxylic esters, in particular the methyl esters and the ethyl esters.

The pyridazinecarboxylic acids of the general formula (II) and derivatives thereof as required starting materials have hitherto not been disclosed in the literature; as novel compounds, they also form part of the subject matter of the present application.

The novel pyridazinecarboxylic acids of the formula (II) are obtained when pyridazines of the formula (IV)

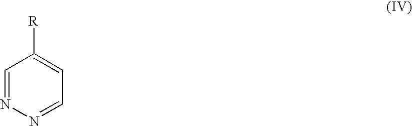

in which R is as defined above are, in a first reaction step, reacted with methyl pyruvate of the formula (V)

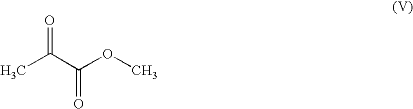

in the presence of hydrogen peroxide (H₂O₂), in the presence of an acid, such as, for example, sulfuric acid, in the presence of a further reaction auxiliary, such as, for example, iron(II) sulfate heptahydrate, and in the presence of diluents, such as, for example, dichloromethane and water, at temperatures between −40° C. and +20° C., and worked up in a customary manner, and the resulting novel pyridazinecarboxylic esters of the formula (IIa),

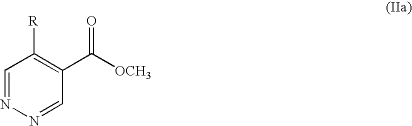

in which R is as defined above are, in a second reaction step, hydrolyzed, for example by reaction with water in the presence of a strong base, such as, for example, sodium hydroxide, and, if appropriate, in the presence of an organic diluent, such as, for example, tetrahydrofuran, at temperatures between 0° C. and 50° C., and worked up in a customary manner.

The resulting novel pyridazinecarboxylic acids of the formula (IIb)

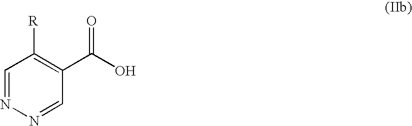

in which R is as defined above can be used to prepare corresponding novel thiono or imino derivatives (Q¹=S, Q¹=N—R¹) by customary methods.

Pyridazinecarbonyl chlorides of the formula (IIc)

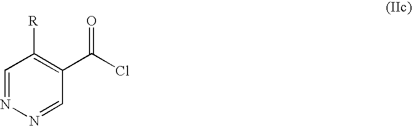

in which R is as defined above, as preferred reactive novel derivatives, can be prepared from the pyridazinecarboxylic acids of the formula (IIb) in a customary manner, for example by reaction with thionyl chloride, at temperatures between 50-100° C.

The pyridazines of the formula (IV) to be used as starting materials are known and/or can be prepared by processes known per se (cf. Rec. Trav. Chim. Pays-Bas 109 (1990), 577-582—cited in Chem. Abstracts 114:122237).

The formula (III) provides a general definition of the substituted nitrogen compounds further to be used as starting materials in the process (a) according to the invention for preparing the compounds of the general formula (I). In the general formula (III), Q² preferably or in particular has that meaning which has already been mentioned above, in connection with the description of the compounds of the general formula (I) according to the invention, as being preferred or as being particularly preferred for Q².

The starting materials of the general formula (III) are known and/or can be prepared by processes known per se.

The process (a) according to the invention for preparing the compounds of the general formula (I) is preferably carried out using one or more reaction auxiliaries. Reaction auxiliaries suitable for the process according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, cesium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexyl-amine, dicyclohexylamine, ethyldicyclohexylamine, N,N-dimethylaniline, N,N-dimethylbenzyl-amine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-Diazabicyclo[4.3.0]non-5-ene (DBN), or 1,8-diazabicyclo[5.4.0]undec-7-ene (DBU).

The process (a) according to the invention for preparing the compounds of the general formula (I) is preferably carried out using one or more diluents. Suitable diluents for carrying out the process (a) according to the invention are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, disopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-di-methylacetamide, N-methylformanilide, N-methylpyrrolidone or hexamethylphosphoric triamide; esters, such as methyl acetate or ethyl acetate, sulfoxides, such as dimethyl sulfoxide, alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the process (a) according to the invention, the reaction temperatures can be varied within a relatively wide range. In general, the process is carried out at temperatures between −20° C. and +150° C., preferably between 0° C. and 100° C.

The process (a) according to the invention is generally carried out under atmospheric pressure. However, it is also possible to carry out the process according to the invention under elevated or reduced pressure—in general between 0.1 bar and 10 bar.

The novel substituted pyridazinecarboxamides and derivatives thereof of the formula (I) have multifarious biological properties; they can be used especially as plant treatment agents, but also for other areas of use, in particular for controlling animal pests, very particularly insects, of importance in agriculture, in forests, in the protection of stored products and materials, in the hygiene sector and in the sector of animal health.

The compounds of the formula (I) according to the invention are well tolerated by plants, have favorable homeotherm toxicity and are environmentally friendly; they are suitable for protecting plants and plant organs, for increasing harvest yields, for improving the quality of the harvested material and for controlling animal pests, in particular insects, arachnids and nematodes encountered in agriculture, in forests, in gardens and leisure facilities, in the protection of stored products and materials and in the hygiene sector. They may preferably be used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregana.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The compounds of the formula (I) according to the invention are suitable in particular for controlling sucking insects, such as aphids (for example Aphis fabae, Aphis pomi, Aphis spiraecola, Aphis gossypii, Aphis nasturtii, Dysaphis plantaginea, Eriosoma spp., Rhopalosiphum padi, Acyrthosiphon pisum, Pemphigus bursarius, Myzus persicae, Myzus nicotianae, Myzus euphorbiae, Phylloxera spp., Toxoptera spp., Brevicoryne brassicae, Macrosiphum avenae, Macrosiphum euphorbiae, Nasonovia ribisnigri, Sitobion avenae, Brachycaudius helychrysii, Phorodon humuli), leaf hoppers (for example Idioscopus clypealis, Scaphoides titanus, Empoasca onuki, Empoasca vitis, Empoasca devastans, Empoasca libyca, Empoasca biguttula, Empoasca facialis, Erythroneura spp.), thrips (Hercinothrips femoralis, Scirtothrips aurantii, Scirtothrips dorsalis, Frankliniella spp., Kakothrips spp., Thrips oryzae, Thrips palmi, Thrips tabaci) or white flies (Aleurodes brassicae, Bemisia tabaci, Trialeurodes vaporariorum, Aleurodes proletella).

If appropriate, the compounds according to the invention may also be used in certain concentrations or application rates as herbicides or as safeners for them, or as microbicides, for example as fungicides, antimycotics and bactericides. If appropriate, they can also be employed as intermediates or precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by varietal property rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

The treatment according to the invention of the plants and plant parts with the active compounds is carried out directly or by allowing the compounds to act on their surroundings, environment or storage space by the customary treatment methods, for example by immersion, spraying, evaporation, fogging, scattering, spreading or injecting and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are: for example ammonium salts and ground natural minerals such as kaolins, clays, talc, chalk, quartz, attapulgite, montmorillonite or diatomaceous earth, and ground synthetic materials such as highly-disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally contain between 0.1% and 95% by weight of active compound, preferably between 0.5% and 90%.

The active compounds according to the invention may be present in their commercially customary formulations, and in the application forms prepared from these formulations, as mixtures with other active compounds, such as insecticides, attractants, sterilants, bactericides, acaricides, nematicides, fungicides, growth regulators or herbicides. The insecticides include, for example, phosphates, carbamates, carboxylates, chlorinated hydrocarbons, phenylureas, substances produced by microorganisms, etc.

Particularly favorable cocomponents in the mixtures are, for example, the following:

Fungicides: 2-phenylphenol; 8-hydroxyquinoline sulfate; acibenzolar-5-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chloro-phenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro[4.5]decane-3-amine; sodium tetrathiocarbonate;
and copper salts and preparations, such as Bordeaux mixture; copper hydroxide; copper naphthenate; copper oxychloride; copper sulfate; cufraneb; cuprous oxide; mancopper; oxine-copper.
Bactericides: bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/acaricides/nematicides: abamectin, ABG-9008, acephate, acequinocyl, acetamiprid, acetoprole, acrinathrin, AKD-1022, AKD-3059, AKD-3088, alanycarb, aldicarb, aldoxycarb, allethrin, alpha-cypermethrin (alphamethrin), amidoflumet, aminocarb, amitraz, avermectin, AZ-60541, azadirachtin, azamethiphos, azinphos-methyl, azinphos-ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, benclothiaz, bendiocarb, benfuracarb, bensultap, benzoximate, beta-cyfluthrin, beta-cypermethrin, bifenazate, bifenthrin, binapacryl, bioallethrin, bioallethrin-5-cyclopentyl-isomer, bioethanomethrin, biopermethrin, bioresmethrin, bistrifluoron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben, cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, cyfluthrin, cyhalothrin, cyhexatin, cypermethrin, cyphenothrin (1R-trans-isomer), cyromazine, DDT, deltamethrin, demeton-S-methyl, demeton-S-methylsulfone, diafenthiuron, dialifos, diazinon, dichlofenthion, dichlorvos, dicofol, dicrotophos, dicyclanil, diflubenzuron, dimefluthrin, dimethoate, dimethylvinphos, dinobuton, dinocap, dinotefuran, diofenolan, disulfoton, docusat-sodium, dofenapyn, DOWCO-439, eflusilanate, emamectin, emamectin-benzoate, empenthrin (1R-isomer), endosulfan, Entomopthora spp., EPN, esfenvalerate, ethiofencarb, ethiprole, ethion, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufen-zine), fluvalinate, fonofos, formetanate, formothion, fosmethilan, fosthiazate, fubfenprox (fluproxyfen), furathiocarb, gamma-cyralothrin, gamma-HCH, gossyplure, grandlure, granulosis viruses, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda-cyhalothrin, lindane, lufenuron, malathion, mecarbam, mesulfenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metofluthrin, metolcarb, metoxadiazone, mevinphos, milbemectin, milbemycin, MKI-245, MON-45700, monocrotophos, moxidectin, MTI-800, naled, NC-104, NC-170, NC-184, NC-194, NC-196, niclosamide, nicotine, niten-pyram, nithiazine, NNI-0001, NNI-0101, NNI-0250, NNI-9768, novaluron, noviflumuron, OK-5101, OK-5201, OK-9601, OK-9602, OK-9701, OK-9802, omethoate, oxamyl, oxydemeton-methyl, Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, potassium oleate, prallethrin, profenofos, prefluthrin, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen, quinalphos, resmethrin, RH-5849, ribavirin, RU-12457, RU-15525, S-421, S-1833, salithion, sebufos, SI-0009, silafluofen, spinosad, spirodiclofen, spiromesifen, sulfluramid, sulfotep, sulprofos, SZI-121, tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogen oxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, Trichoderma atroviride, triflumuron, trimethacarb, vamidothion, vaniliprole, verbutin, Verticillium lecanii, WL-108477, WL-40027, yl-5201, yl-5301, yl-5302, XMC, xylylcarb, ZA-3274, zeta-cypermethrin, zolaprofos, ZXI-8901, the compound 3-methylphenyl propylcarbamate (tsumacide Z), the compound 3-(5-chloro-3-pyridinyl)-8-(2,2,2-trifluoroethyl)-8-azabicyclo[3.2.1]octane-3-carbonitrile (CAS-Reg. No. 185982-80-3) and the corresponding 3-endo-isomer (CAS-Reg. No. 185984-60-5) (cf. WO-96/37494, WO-98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

When used as insecticides in their commercially available formulations and in the use forms prepared with these formulations, the active compounds according to the invention can furthermore be present in the form of a mixture with synergists. Synergists are compounds by which the activity of the active compounds is increased without it being necessary for the synergist added to be active itself.

When used as insecticides in their commercially available formulations and in the use forms prepared with these formulations, the active compounds according to the invention can furthermore be present in the form of a mixture with inhibitors which reduce the degradation of the active compound after application in the habitat of the plant, on the surface of parts of plants or in plant tissues.

The active compound content of the use forms prepared from the commercially available formulations can vary within broad ranges. The active compound concentration of the use forms can be from 0.0000001% up to 95% by weight of active compound, preferably between 0.0001 and 1% by weight.

They are applied in a customary manner adapted to suit the use forms.

When used against hygiene pests and pests of stored products, the active compound is distinguished by excellent residual action on wood and clay as well as good stability to alkali on limed substrates.

As already mentioned above, it is possible to treat all plants or their parts in accordance with the invention. In a preferred embodiment, wild plant species or plant varieties and plant cultivars which have been obtained by traditional biological breeding methods, such as hybridization or protoplast fusion, and the parts of these varieties and cultivars are treated. In a further preferred embodiment, transgenic plants and plant cultivars which have been obtained by recombinant methods, if appropriate in combination with conventional methods (genetic modified organisms), and their parts are treated. The term “parts” or “parts of plants” or “plant parts” has been explained above.

Plants which are treated particularly preferably in accordance with the invention are those of the plant cultivars which are in each case commercially available or in use. Plant cultivars are understood as meaning plants with new traits which have been bred either by conventional breeding, by mutagenesis or by recombinant DNA techniques. They may take the form of cultivars, biotypes and genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or a widened activity spectrum and/or an increase in the activity of the substances and compositions which can be used in accordance with the invention, better plant growth, increased tolerance to high or low temperatures, increased tolerance to drought or to salinity in the water or soil, increased flowering performance, facilitated harvesting, accelerated maturation, higher yields, higher quality and/or better nutritional value of the harvested products, better storage characteristics and/or processability of the harvested products are possible which exceed the effects which were actually to be expected.

The preferred transgenic plants or plant cultivars (those obtained by recombinant methods) to be treated in accordance with the invention include all those plants which, owing to the process of recombinant modification, were given genetic material which confers particular, advantageous, valuable traits to these plants. Examples of such properties are better plant growth, increased tolerance to high or low temperatures, increased tolerance to drought or to salinity in the water or soil, increased flowering performance, facilitated harvesting, accelerated maturation, higher yields, higher quality and/or higher nutritional value of the harvested products, better storage characteristics and/or better processability of the harvested products. Further examples of such traits, examples which must be mentioned especially, are better defense of the plants against animal and microbial pests, such as against insects, mites, phytopathogenic fungi, bacteria and/or viruses and an increased tolerance of the plants to certain herbicidal active compounds. Examples of transgenic plants which may be mentioned are the important crop plants, such as cereals (wheat, rice), maize, soybeans, potato, cotton, tobacco, oilseed rape and fruit plants (with the fruits apples, pears, citrus fruits and grapes), with particular emphasis on maize, soybeans, potatoes, cotton tobacco, and oilseed rape. Traits which are especially emphasized are the increased defense of the plants against insects, arachnids, nematodes, and slugs and snails owing to toxins being formed in the plants, in particular toxins which are generated in the plants by the genetic material of Bacillus thuringiensis (for example by the genes CryIA(a), CryIA(b), CryIA(c), CryIIA, CryIIIA, CryIIIB2, Cry9c Cry2Ab, Cry3Bb and CryIF and their combinations; hereinbelow “Bt plants”). Other traits which are particularly emphasized are the increased defense of plants against fungi, bacteria and viruses by the systemic acquired resistance (SAR), systemin, phytoalexins, elicitors and resistance genes and correspondingly expressed proteins and toxins. Other traits which are especially emphasized are the increased tolerance of the plants to certain herbicidal active compounds, for example imidazolinones, sulfonylureas, glyphosate or phosphinotricin (for example “PAT” gene). The genes which confer the desired traits in each case may also be present in the transgenic plants in combination with one another. Examples of “Bt plants” which may be mentioned are maize cultivars, cotton cultivars, soybean cultivars and potato cultivars which are commercially available under the trade names YIELD GARD® (for example maize, cotton, soybeans), KnockOut® (for example maize), StarLink® (for example maize), Bollgard® (cotton), Nucotn® (cotton) and NewLeaf® (potato). Examples of herbicide-tolerant plants which may be mentioned are maize cultivars, cotton cultivars and soybean cultivars which are commercially available under the trade names Roundup Ready® (tolerance to glyphosate, for example maize, cotton, soybean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned include also the varieties commercially available under the name Clearfield® (for example maize). Naturally, these statements also apply to plant cultivars having these genetic traits or genetic traits still to be developed, which plant cultivars will be developed and/or marketed in the future.

The plants listed can be treated particularly advantageously according to the invention with the compounds of the general formula I or the active compound mixtures according to the invention. The preferred ranges stated above for the active compounds and mixtures also apply to the treatment of these plants. Particular emphasis may be given to the treatment of plants with the compounds or mixtures specifically mentioned in the present text.

The active compounds according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and sucking), parasitic fly larvae, lice, hair lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the sub-orders Amblycerina and Ischnocerina, for example, Trimenopon spp., Menopon spp., Trinoton spp., Bovicola spp., Wemeckiella spp., Lepikentron spp., Damalina spp., Trichodectes spp., Felicola spp.

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the sub-class of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea-pigs, rats and mice. By combating these arthropods, it is intended to reduce deaths and decreased performances (in meat, milk, wool, hides, eggs, honey and the like), so that more economical and simpler animal keeping is made possible by using the active compounds according to the invention.

In the veterinary sector, the active compounds according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds of the formula (I) can be used as formulations (for example powders, emulsions, flowables) which comprise the active compounds in an amount of 1 to 80% by weight, either directly or after dilution by a factor of 100 to 10 000, or they may be used in the form of a chemical bath.

Furthermore, it has been found that the compounds according to the invention have a potent insecticidal action against insects which destroy industrial materials.

The following insects may be mentioned by way of example and as being preferred, but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus;

Dermapterans, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example:

construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flash point of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of terpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flash point of above 30° C., preferably above 45° C., can be partially replaced by organochemical solvents of high or medium volatility, with the proviso that the solvent mixture also has an evaporation number of above 35 and a flash point of above 30° C., preferably above 45° C., and that the insecticide/fungicide mixture is soluble or emulsifiable in this solvent mixture.

In a preferred embodiment, part of the organochemical solvent or solvent mixture or an aliphatic polar organochemical solvent or solvent mixture is replaced. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/cumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odor-masking substances and inhibitors or anticorrosives known per se and the like can also be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the abovementioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di-(2-ethylhexyl)adipate, the stearates, such as butyl stearate or amyl stearate, the oleates, such as butyl oleate, the glycerol ethers or relatively high-molecular-weight glycol ethers, glycerol esters and p-toluenesulfonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the abovementioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxid, triflumuron, chlothianidin, spinosad, tefluthrin,

and fungicides, such as epoxyconazole, hexaconazole, azaconazole, propiconazole, tebuconazole, cyproconazole, metconazole, imazalil, dichlofluanid, tolylfluanid, 3-iodo-2-propynylbutyl carbamate, N-octylisothiazolin-3-one and 4,5-dichloro-N-octylisothiazolin-3-one.

The compounds according to the invention can at the same time be employed for protecting objects which come into contact with saltwater or brackish water, in particular hulls, screens, nets, buildings, moorings and signaling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Using the compounds according to the invention, alone or in combination with other active compounds, allows the use of heavy metals such as, for example, in bis(trialkyltin) sulfides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulfide, antimony oxide, polymeric butyl titanate, phenyl(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole;
molluscicides such as fentin acetate, metaldehyde, methiocarb, niclosamid, thiodicarb and trimethacarb; Fe chelates,
or conventional antifouling active compounds such as 4,5-dichloro-2-octyl-4-isothiazolin-3-one, diiodomethylparatryl sulfone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulfonyl)pyridine, 2,4,5,6-tetrachloro-isophthalonitrile, tetramethylthiuram disulfide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in saltwater. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalis, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, unpressurized spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, unenergized, or passive, evaporation systems, moth papers, moth bags and moth gels, as granules or dusts, in baits for spreading or in bait stations.

### PREPARATION EXAMPLES

### Example 1

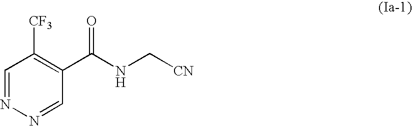

Process (a): 175.8 mg (1.90 mmol) of aminoacetonitrile hydrochloride and 288 mg (2.85 mmol) of triethylamine are added to 3 ml of 1,4-dioxane and stirred at room temperature (about 20° C.) for 3 hours. A solution of 200 mg (0.95 mmol) of 5-trifluoromethylpyridazine-4-carbonyl chloride in 1 ml of 1,4-dioxane is then added dropwise, and the reaction mixture is stirred at room temperature overnight. The reaction solution is concentrated and the dark brown residue is purified by chromatography on silica gel using cyclohexane/ethyl acetate 2:1 as mobile phase.

This gives 73 mg (31% of theory) of N-cyanomethyl-5-trifluoromethylpyridazine-4-carboxamide, HPLC: log P (pH 2.3)=0.60.

Analogously to preparation example 1 and in accordance with the general description of the preparation processes according to the invention, it is also possible to prepare, for example, the compounds of the formula (I)—or the formulae (Ia) and (Ib)—listed in tables 1 and 2 below.

TABLE 1
Examples of the compounds of the formula (Ia)

| | |
|---|---|
| | (Ia) |
| 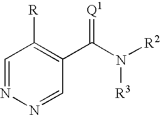 | |

| | | | | | Physical |
|---|---|---|---|---|---|
| Ex. No. | R | Q¹ | R² | R³ | data |
| Ia-2 | CF₃ | O | H | H | (oil) |
| Ia-3 | CF₃ | O | H | CH₃ | |
| Ia-4 | CF₃ | O | H | C₂H₅ | |
| Ia-5 | CF₃ | O | CH₃ | CH₂CN | |
| Ia-6 | CF₃ | O | C₂H₅ | CH₂CN | |
| Ia-7 | CF₃ | O | CH₂OCH₃ | CH₂CN | |
| Ia-8 | CF₃ | O | CH₂COOC₂H₅ | CH₂CN | |
| Ia-9 | CF₃ | O | CH₂CH₂OH | CH₂CN | |
| Ia-10 | CF₃ | O | (CO)C₆H₅ | CH₂CN | |
| Ia-11 | CF₃ | S | H | H | |
| Ia-12 | CF₃ | S | CH₃ | CH₃ | |
| Ia-13 | CF₃ | O | H | CH₂OCH₃ | |
| Ia-14 | CF₃ | O | (CO)CH₃ | CH₂CN | |
| Ia-15 | CF₃ | O | CH₂(CO)CH₃ | CH₂CN | |
| Ia-16 | CF₃ | O | H | CH₂OH | |
| Ia-17 | CF₃ | O | H | COOC₂H₅ | |
| Ia-18 | CF₃ | O | H | CH₂COOC₂H₅ | |
| Ia-19 | CF₃ | O | H | CH₂CH═CH₂ | |
| Ia-20 | CF₃ | O | H | CH₂CH₂OCH₃ | |
| Ia-21 | CF₃ | O | H | CH₂CH₂CN | |
| Ia-22 | CF₃ | O | H | CH₂CH₂OH | |
| Ia-23 | CF₃ | O | CH₂(CO)C₆H₅ | CH₂CN | |
| Ia-24 | CF₃ | O | SO₂CH₃ | CH₂CN | |
| Ia-25 | CF₃ | O | SO₂C₆H₅ | CH₂CN | |
| Ia-26 | CF₃ | O | CH₂C₆H₅ | CH₂CN | |
| Ia-27 | CF₃ | O | H | OCH₃ | |
| Ia-28 | CF₃ | O | CH₃ | OCH₃ | |
| Ia-29 | CF₃ | O | H | OC₂H₅ | |
| Ia-30 | CF₃ | O | CH₂CH═CH₂ | CH₂CN | |
| Ia-31 | CF₃ | O | CH₃ | CH₂CN | |
| Ia-32 | CF₃ | O | CH₂CH₂OCH₃ | CH₂CN | |
| Ia-33 | CF₃ | O | COOC₂H₅ | CH₂CN | |
| Ia-34 | CF₃ | O | CH₃ | CH₂CH₃ | logP = 1.15^{a)} |
| Ia-35 | CF₃ | O | CH₃ | (CH₂)₂CH₃ | logP = 1.62^{a)} |
| Ia-36 | CF₃ | O | CH₃ | CH₂CH(CH₃)₂ | logP = 1.96^{a)} |
| Ia-37 | CF₃ | O | H | CH(CH₃)CH₂SCH₃ | logP = 1.53^{a)} |
| Ia-38 | CF₃ | O | H | (CH₂)₂OH | |
| Ia-39 | CF₃ | O | H | CH₂CH(CH₃)OH | logP = 0.43^{a)} |
| Ia-40 | CF₃ | O | H | CH₂COCH₃ | |
| Ia-41 | CF₃ | O | H | CH₂CH═NOCH₃ | |
| Ia-42 | CF₃ | O | H | CH₂CH═NOC₂H₅ | |
| Ia-43 | CF₃ | O | H | CH₂C(CH₃)═NOCH₂Ph | |
| Ia-44 | CF₃ | O | H | CH₂C(CH₃)═NOCH₂ | |
| | | | | (p-Tol.) | |
| Ia-45 | CF₃ | O | H | 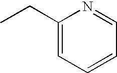 | logP = 0.11^{a)} |
| Ia-46 | CF₃ | O | H | 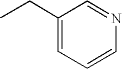 | logP = 0.12^{a)} |
| Ia-47 | CF₃ | O | H | 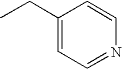 | logP = 1.98^{a)} |
| Ia-48 | CF₃ | O | H | 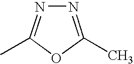 | logP = 1.47^{a)} |
| Ia-49 | CF₃ | O | H | 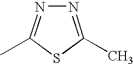 | |
| Ia-50 | CF₃ | O | H | 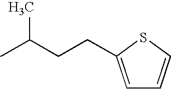 | |
| Ia-51 | CF₃ | O | H | 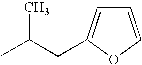 | logP = 1.86^{a)} |
| Ia-52 | CF₃ | O | H | 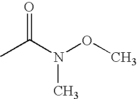 | |
| Ia-53 | CF₃ | O | H | COOCH₃ | |
| Ia-54 | CF₃ | O | H | COOCH₂C₆H₅ | |
| Ia-55 | CF₃ | O | H | 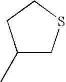 | |
| Ia-56 | CF₃ | O | H | 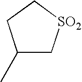 | |
| Ia-57 | CF₃ | O | H | 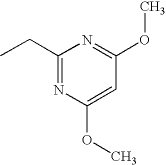 | m.p.: 151° C. |
| Ia-58 | CF₃ | O | H | 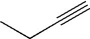 | |
| Ia-59 | CF₃ | O | H | 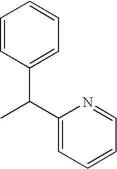 | |

The log P values given in the table were determined in accordance with EEC directive 79/831 Annex V.A8 by HPLC (High Performance Liquid Chromatography) on a reverse-phase column (C 18). Temperature: 43° C.

(a) Mobile phases for the determination in the acidic range (pH 2.3): 0.1% aqueous phosphoric acid, acetonitrile; linear gradient from 10% acetonitrile to 90% acetonitrile—the corresponding measurement results in table 1 are marked a).

(b) Mobile phases for the determination in the neutral range (pH 7.5): 0.01-molar aqueous phosphate buffer solution, acetonitrile; linear gradient from 10% acetonitrile to 90% acetonitrile—the corresponding measurement results in table 1 are marked b).

Calibration was carried out using unbranched alkan-2-ones (having 3 to 16 carbon atoms) with known log P values (determination of the log P values by the retention times using linear inter-polation between two successive alkanones).

The lambda max values were determined in the maxima of the chromatographic signals using the UV spectra from 200 nm to 400 nm.

TABLE 2
Examples of compounds of the formula (Ib)

| | |
|---|---|
| | (Ib) |
| 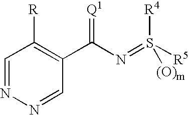 | |

| | | | R⁴ or | | | Physical |
|---|---|---|---|---|---|---|
| Ex. No. | R | Q¹ | R⁴ + R⁵ | R⁵ | m | data |
| Ib-1 | CF₃ | O | CH₃ | CH₃ | 0 | |
| Ib-2 | CF₃ | O | CH₃ | CH₃ | 1 | |
| Ib-3 | CF₃ | O | C₂H₅ | C₂H₅ | 0 | |
| Ib-4 | CF₃ | O | C₂H₅ | C₂H₅ | 1 | |
| Ib-5 | CF₃ | O | n-C₃H₇ | n-C₃H₇ | 0 | |
| Ib-6 | CF₃ | O | n-C₃H₇ | n-C₃H₇ | 1 | |
| Ib-7 | CF₃ | O | i-C₃H₇ | i-C₃H₇ | 0 | |
| Ib-8 | CF₃ | O | C₆H₅ | C₆H₅ | 0 | |
| Ib-9 | CF₃ | O | C₆H₅ | C₆H₅ | 1 | |
| Ib-10 | CF₃ | O | n-C₄H₉ | n-C₄H₉ | 0 | |
| Ib-11 | CF₃ | O | (CH₂)₄ | — | 0 | |
| Ib-12 | CF₃ | O | CH₃ | 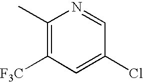 | 0 | |

### Starting Compounds of the Formula (II):

### Example II-1

### Step 1

At about 0° C., 2.3 g (20.26 mmol) of hydrogen peroxide are added dropwise to 3.1 g (30.39 mmol) of methyl pyruvate, and the solution obtained in this manner is, also at about 0° C., added dropwise to a mixture of 1.0 g (6.75 mmol) of 4-trifluoromethylpyridazine, 2.0 g of conc. sulfuric acid, 6 ml of water and 5.6 g (20.29 mmol) of iron(II) sulfate heptahydrate in 30 ml of dichloromethane. The reaction mixture is stirred for another 15 minutes and then poured into 80 ml of water. The organic phase is separated off, washed once with water and stirred with 60 ml of 10% strength bisulfite solution for 30 minutes. The organic phase is removed, again washed with water and dried over sodium sulfate, and the solvent is distilled off. The crude product is purified by chromatography on silica gel using the mobile phase cyclohexane/ethyl acetate 2:1. This gives 1.1 g (70% of theory) of methyl 5-trifluoromethylpyridazine-4-carboxylate as a colorless oil. HPLC: log P (pH 2.3)=1.68

### Step 2

2.0 g (9.70 mmol) of methyl 5-trifluoromethylpyridazine-4-carboxylate are dissolved in a mixture of 15 ml each of tetrahydrofuran and water, and 14.5 ml (29.1 mmol) of 2N aqueous sodium hydroxide solution is added dropwise. After 60 minutes, the mixture is adjusted to pH 1 using semiconcentrated hydrochloric acid, is diluted with 20 ml of water and is extracted 3× with methyl tert-butyl ether. The organic phases are combined and dried over sodium sulfate, and the solvent is distilled off. This gives 1.4 g (75% of theory) of 5-trifluoromethylpyridazine-4-carboxylic acid as a white solid. m.p.: 150-51° C.

### Example II-2

At room temperature (about 20° C.), 0.4 g (2.08 mmol) of 5-trifluoromethylpyridazine-4-carboxylic acid is added to 2.48 g (20.8 mmol) of thionyl chloride. The mixture is slowly heated to 80° C. and stirred at this temperature for 30 minutes. Excess thionyl chloride is then distilled off, and twice, toluene is added to the residue, which is in each case re-concentrated. This gives 5-trifluoromethylpyridazine-4-carbonyl chloride as an orange oil which is used without further purification for the next step (cf. example 1). Yield: 0.435 g (99% of theory)

### Example A

### Phaedon Test

### Spray Treatment

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of Chinese cabbage (Brassica pekinensis) are sprayed with a preparation of active compound of the desired concentration and, after drying, populated with larvae of the mustard beetle (Phaedon cochleariae).

After the desired period of time, the effect in % is determined. 100% means that all beetle larvae have been killed; 0% means that none of the beetle larvae have been killed.

In this test, for example, the following compound of the preparation examples shows good activity:

TABLE A
Plant-damaging insects
Phaedon test (spray treatment)

| | | Active compound | Kill rate |
|---|---|---|---|
| Active compounds | | concentrations in g/ha | in % after 7^{d} |
| Ex. Ia-59 | 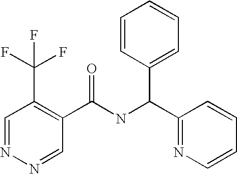 | 500 | 100 |

### Example B

### Spodoptera frugiperda Test

### Spray Treatment

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of corn leaves (Zea mays) are sprayed with a preparation of active compound of the desired concentration and, after drying, populated with caterpillars of the armyworm (Spodoptera frugiperda).

After the desired period of time, the effect in % is determined. 100% means that all caterpillars have been killed; 0% means that none of the caterpillars have been killed.

In this test, for example, the following compound of the preparation examples shows good activity:

TABLE B
Plant-damaging insects
Spodoptera frugiperda test (spray treatment)

| | | Active compound | Kill rate |
|---|---|---|---|
| Active compounds | | concentration in g/ha | in % after 7^{d} |
| Ex. Ia-59 | 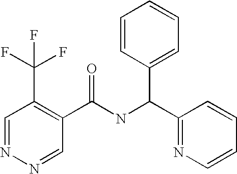 | 500 | 100 |

1. A compound of the formula (I) 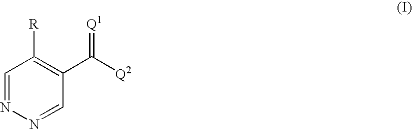
in which
R represents halogen-substituted alkyl,
Q¹ represents O (oxygen), S (sulfur) or the grouping N—R¹,
Q² represents one of the groupings below 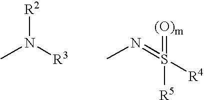
R¹ represents hydrogen, hydroxyl, or in each case optionally substituted alkyl, alkylcarbonyl, alkoxy, alkoxycarbonyl, alkylcarbonyloxy, alkoxycarbonyloxy, alkylsulfonyl, alkylsulfonyloxy, alkenyl, alkynyl, alkenyloxy, alkynyloxy, cycloalkyl, cycloalkyloxy, cycloalkylalkyl, cycloalkylalkoxy, cycloalkylcarbonyl, cycloalkylsulfonyl, cycloalkylsulfonyloxy, aryl, aryloxy, arylalkyl, arylalkoxy, arylcarbonyl, arylsulfonyl, aryl-sulfonyloxy, heterocyclyl, heterocyclylalkyl, heterocyclylcarbonyl, heterocyclylsulfonyl or heterocyclylsulfonyloxy,
R² represents one of the groupings -A¹-R⁶ or -A²-O—R⁶— or -A¹-S(O)ₙ—R⁶ or -A²-N(R⁶)₂ or -A²-N(R⁶)—O—R⁶ or -A²-O—N(R⁶)₂ or -A²-O—N═C(R⁶)₂ or -A²-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A²-(C=Q³)-N(R⁶)₂ or -A²-(C=Q³)-N(R⁶)OR⁶ or -A²-O—(C=Q³)-R⁶ or -A²-N(R⁶)—(C=Q³)-R⁶ or -A²-C(R⁶)═N—O—R⁶ or -A²-C(R⁶)═N(R⁶)—N(R⁶)₂ or -A²-C(R⁶)═N(R⁶)—N(R⁶)—(C=Q³)-(Q³)ₘ—R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different, and
R³ represents one of the groupings -A¹-R⁶ or -A¹-O—R⁶— or -A¹-S(O), —R⁶ or -A¹-N(R⁶)₂ or -A²-N(R⁶)—O—R⁶ or -A²-O—N(R⁶)₂ or -A²-O—N═C(R⁶)₂ or -A¹-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —N(R⁶)-A¹-(C=Q³)-R⁶ or -A¹-N(R⁶)—(C=Q³)-R⁶ or -A¹-C(R⁶)═N—O—R⁶ or -A¹-C(R⁶)═N(R⁶)—N(R⁶)₂ or -A²-C(R⁶)═N(R⁶)—N(R⁶)—(C=Q³)-(Q³)-R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different, or
R³ and R² together represent the grouping ═CR⁷R⁸, or
R¹ and R² together with the nitrogen atom to which the two groups are attached form an optionally substituted and optionally benzo-fused heterocycle which optionally contains one to three further nitrogen atoms and/or one oxygen atom and/or one (SO)ₙ-grouping and optionally additionally one (C=Q¹) grouping, or
R³ and R² together represents the grouping ═P(X)₃, where X represents amino, alkyl, alkylamino, cycloalkyl, cycloalkylamino, phenyl or phenylamino and where the radicals X may in each case be identical or different, and
R⁴ represents in each case optionally substituted alkyl, alkoxy, alkylamino, dialkylamino, alkenyl, alkynyl, cycloalkyl, cycloalkylamino, N-alkyl-cycloalkylamino, cycloalkenyl, cycloalkylalkyl, aryl, arylamino, N-alkyl-arylamino, arylalkyl, arylalkylamino, N-alkyl-arylalkylamino, N-cycloalkyl-arylalkylamino, heterocyclyl or heterocyclylalkyl, and
R⁵ represents in each case optionally substituted alkyl, alkoxy, alkylamino, dialkylamino, alkenyl, alkynyl, cycloalkyl, cycloalkylamino, N-alkyl-cycloalkylamino, cycloalkenyl, cycloalkylalkyl, aryl, arylamino, N-alkyl-arylamino, arylalkyl, arylalkylamino, N-alkyl-arylalkylamino, N-cycloalkyl-arylalkylamino, heterocyclyl or heterocyclylalkyl, or together with R⁴ represents optionally substituted and/or benzo-fused alkanediyl (alkylene), alkyleneamino or alkylenediamino, each of which is optionally interrupted by O (oxygen) and/or N (nitrogen) and/or an S(O)ₙ grouping and/or a (C=Q¹)-grouping,
R⁶ represents hydrogen, hydroxyl, amino, cyano or in each case optionally substituted alkyl, alkenyl, alkynyl, cycloalkyl, cycloalkenyl, cycloalkylalkyl, cycloalkenylalkyl, aryl, arylalkyl, heterocyclyl or heterocyclylalkyl, or—if two groupings R⁶ are located in a geminal position on a C═C double bond—together they also represent alkanediyl (alkylene), where one hydrocarbon component of the alkanediyl grouping may be replaced by O (oxygen) and/or N (nitrogen) and/or an S(O)ₙ grouping and/or a (C=Q¹) grouping,
R⁷ represents in each case optionally substituted alkoxy, alkylthio, alkylamino, dialkylamino, alkenyloxy, alkenylthio, alkenylamino, alkynyloxy, alkynylthio, alkynylamino, cycloalkyloxy, cycloalkylthio, cycloalkylamino, cycloalkylalkoxy, cycloalkylalkylthio, cycloalkylalkylamino, arylalkoxy, arylalkylthio or arylalkylamino,
R⁸ represents in each case optionally substituted alkoxy, alkylamino, dialkylamino, alkenyloxy, alkenylthio, alkenylamino, alkynyloxy, alkynylthio, alkynylamino, cycloalkyloxy, cycloalkylthio, cycloalkylamino, cycloalkylalkoxy, cycloalkylalkylthio, cycloalkylalkylamino, arylalkoxy, arylalkylthio or arylalkylamino, or together with R⁷ represents in each case optionally substituted thiaalkyleneoxy, thiaalkyleneamino, thiaalkenyleneamino, alkylenediamino or alkenylenediamino in which optionally one methylene grouping is replaced by O (oxygen), S (sulfur) or N (nitrogen),
m represents the number 0 or 1,
n represents the number 0, 1 or 2,
A¹ represents a single bond or optionally substituted alkanediyl (alkylene),
A² represents optionally substituted alkanediyl (alkylene), and
Q³ represents O (oxygen) or S (sulfur).

2. The compound of the formula (I) as claimed in claim 1 in which
R represents alkyl having 1 to 6 carbon atoms which is mono- or polysubstituted by halogen,
R¹ represents hydrogen, hydroxyl, in each case optionally hydroxyl-, cyano-, carboxyl-, carbamoyl-, halogen-, C₁-C₆-alkoxy- or C₁-C₆-alkoxycarbonyl-substituted alkyl, alkylcarbonyl, alkoxy, alkoxycarbonyl, alkylcarbonyloxy or alkoxycarbonyloxy having in each case 1 to 8 carbon atoms in the alkyl groups, in each case optionally halogen-substituted alkylsulfonyl or alkylsulfonyloxy having in each case 1 to 8 carbon atoms, in each case optionally cyano-, carboxyl-, carbamoyl-, halogen- or C₁-C₆-alkoxy-carbonyl-substituted alkenyl, alkynyl, alkenyloxy or alkynyloxy having in each case 2 to 8 carbon atoms, in each case optionally cyano-, carboxyl-, carbamoyl-, halogen-, C₁-C₄-alkyl-, C₁-C₄-haloalkyl- or C₁-C₄-alkoxy-carbonyl-substituted cycloalkyl, cycloalkyloxy, cycloalkylalkyl, cycloalkylalkoxy, cycloalkylcarbonyl, cycloalkylsulfonyl or cycloalkylsulfonyloxy having in each case 3 to 6 carbon atoms in the cycloalkyl group and, if appropriate, 1 to 6 carbon atoms in the alkyl moiety, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl, aryloxy, arylalkyl, arylalkoxy, aryl-carbonyl, arylsulfonyl or arylsulfonyloxy having in each case 6 or 10 carbon atoms in the aryl group and, if appropriate, 1 to 6 carbon atoms in the alkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted mono- or bicyclic heterocyclyl, heterocyclylalkyl, heterocyclylcarbonyl, heterocyclylsulfonyl or heterocyclylsulfonyloxy having in each case up to 10 carbon atoms and at least one heteroatom from the group consisting of N (nitrogen), O (oxygen) or an S(O)ₙ grouping and optionally additionally a (C=Q¹) grouping in the heterocyclyl group and, if appropriate, 1 to 6 carbon atoms in the alkyl moiety,
R³ represents one of the groupings -A-R⁶ or -A¹-O—R⁶— or -A¹-S(O)ₙ—R⁶ or -A¹-N(R⁶)₂ or A²-N(R⁶)—O—R⁶ or -A²-O—N(R⁶)₂ or -A²-O—N═C(R⁶)₂ or -A¹-N(R⁶)—S(O)—R⁶ or -A²-N(R)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —N(R⁶)-A¹-(C=Q³)-R⁶ or -A¹-N(R⁶)—(C=Q³)-R⁶ or -A¹-C(R⁶)═N—O—R⁶ or -A¹-C(R⁶)═N(R⁶)—N(R⁶)₂ or -A²-C(R⁶)═N(R⁶)—N(R⁶)—(C=Q³)-(Q³)ₙ-R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different, or
R² and R³ together with the nitrogen atom to which both groups are attached represent an optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted and optionally benzo-fused heterocycle which contains up to 8 carbon atoms and optionally one to three further nitrogen atoms and/or one oxygen atom and/or one S(O)ₙ grouping and optionally additionally one (C=Q¹) grouping, or
R² and R³ represent the grouping ═P(X)₃, where X represents amino, C₁-C₆-alkyl, C₁-C₆-alkylamino, C₃-C₆-cycloalkyl, C₃-C₆-cycloalkylamino, phenyl or phenylamino, where the radicals X may in each case be identical or different, and
R⁴ represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkyl, alkoxy, alkylamino or dialkylamino having in each case 1 to 8 carbon atoms in the alkyl groups, in each case optionally cyano-, carboxyl-, carbamoyl-, thiocarbamoyl-, halogen-, C₁-C₆-alkoxy-carbonyl-, C₁-C₆-alkylamino-carbonyl- or di-(C₁-C₄-alkyl)-amino-carbonyl-substituted alkenyl or alkynyl having in each case 2 to 8 carbon atoms, in each case optionally cyano-, carboxyl-, carbamoyl-, halogen-, C₁-C₄-alkyl- or C₁-C₄-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate, 1 to 6 carbon atoms in the alkyl moiety, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl, arylamino, N-alkyl-arylamino, arylalkyl, arylalkylamino, N-alkyl-arylalkylamino or N-cycloalkyl-arylalkylamino having in each case 6 or 10 carbon atoms in the aryl group and, if appropriate, 1 to 6 carbon atoms in the alkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted monocyclic or bicyclic heterocyclyl or heterocyclylalkyl having in each case up to 10 carbon atoms and at least one heteroatom from the group consisting of N (nitrogen), O (oxygen) or an S(O)ₙ grouping and optionally additionally a (C=Q¹) grouping in the heterocyclyl group and, if appropriate, 1 to 6 carbon atoms in the alkyl moiety,
R⁵ represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkyl, alkoxy, alkylamino or dialkylamino having in each case 1 to 8 carbon atoms in the alkyl groups, in each case optionally cyano-, carboxyl-, carbamoyl-, thiocarbamoyl-, halogen-, C₁-C₆-alkoxy-carbonyl-, C₁-C₆-alkylamino-carbonyl- or di-(C₁-C₄-alkyl)-amino-carbonyl-substituted alkenyl or alkynyl having in each case 2 to 8 carbon atoms, in each case optionally cyano-, carboxyl-, carbamoyl-, halogen-, C₁-C₄-alkyl- or C₁-C₄-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate 1 to 6 carbon atoms in the alkyl moiety, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl, arylamino, N-alkyl-arylamino, arylalkyl, arylalkylamino, N-alkyl-arylalkylamino, N-cycloalkyl-arylalkylamino having in each case 6 or 10 carbon atoms in the aryl group, if appropriate 1 to 6 carbon atoms in the alkyl moiety and if appropriate 3 to 6 carbon atoms in the cycloalkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted monocyclic or bicyclic heterocyclyl or heterocyclylalkyl having in each case up to 10 carbon atoms and at least one heteroatom from the group consisting of N (nitrogen), O (oxygen) or an S(O)ₙ grouping in the heterocyclyl group and, if appropriate 1 to 6 carbon atoms in the alkyl moiety, or together with R⁴ represents in each case optionally cyano-, halogen- or C₁-C₆-alkyl-substituted and/or benzo-fused alkanediyl (alkylene), alkyleneamino or alkylenediamino, each of which has 2 to 6 carbon atoms in the alkylene grouping and is optionally interrupted by O (oxygen) and/or N (nitrogen) and/or an S(O)ₙ grouping and/or a (C=Q¹) grouping,
R⁶ represents hydrogen, hydroxyl, amino, cyano, optionally cyano-, carboxyl-, carbamoyl-, thiocarbamoyl-, hydroxyl-, amino-, halogen-, C₁-C₆-alkoxy-, C₁-C₆-alkylthio-, C₁-C₆-alkylsulfinyl-, C₁-C₆-alkylsulfonyl-, C₁-C₆-alkylamino-, di-(C₁-C₄-alkyl)amino-, C₁-C₆-alkoxy-carbonyl-, C₁-C₆-alkylamino-carbonyl- or di-(C₁-C₄-alkyl)-amino-carbonyl-substituted alkyl having 1 to 8 carbon atoms, in each case optionally cyano-, carboxyl-, carbamoyl-, thiocarbamoyl-, halogen-, C₁-C₆-alkoxy-carbonyl-, C₁-C₆-alkylamino-carbonyl- or di-(C₁-C₄-alkyl)-amino-carbonyl-substituted alkenyl or alkynyl having in each case 2 to 8 carbon atoms, in each case optionally cyano-, carboxyl-, carbamoyl-, halogen-, C₁-C₄-alkyl-, C₁-C₄-haloalkyl- or C₁-C₄-alkoxy-carbonyl-substituted cycloalkyl, cycloalkenyl, cycloalkylalkyl or cycloalkenylalkyl having in each case 3 to 6 carbon atoms in the cycloalkyl or cycloalkenyl groups, and, if appropriate, 1 to 6 carbon atoms in the alkyl moiety, where the cycloalkyl groups each optionally contain a (C=Q¹) grouping, in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted aryl or arylalkyl having in each case 6 or 10 carbon atoms in the aryl groups and, if appropriate, 1 to 6 carbon atoms in the alkyl moiety, or in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted monocyclic or bicyclic heterocyclyl or heterocyclylalkyl having in each case up to 10 carbon atoms and at least one hetero building block from the group consisting of N (nitrogen), O (oxygen), S(O)ₙ and optionally additionally a (C=Q³) grouping in the heterocyclyl group and, if appropriate, 1 to 6 carbon atoms in the alkyl moiety, or, if two groupings R⁶ are located in geminal position on a C═C double bond, these groupings together optionally also represent alkanediyl having 3 to 6 carbon atoms, where one (hydro)carbon unit (C, CH, CH₂) of the alkanediyl grouping may be replaced by O (oxygen), N (nitrogen), an S(O)ₙ grouping or a (C=Q¹) grouping,
R⁷ represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkoxy, alkylthio, alkylamino or dialkylamino having in each case 1 to 8 carbon atoms in the alkyl groups, represents in each case optionally cyano- or halogen-substituted alkenyloxy, alkenylthio, alkenylamino, alkynyloxy, alkynylthio or alkynylamino having in each case 3 to 8 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 4 carbon atoms in the alkyl moiety, or represents in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted arylalkoxy, arylalkylthio or arylalkylamino having in each case 6 or 10 carbon atoms in the aryl group and 1 to 4 carbon atoms in the alkyl moiety,
R⁸ represents in each case optionally cyano-, carboxyl-, carbamoyl-, thiocarbamoyl-, hydroxyl-, amino-, halogen-, C₁-C₆-alkoxy-, C₁-C₆-alkylamino-, di-(C₁-C₄-alkyl)-amino-, C₁-C₆-alkoxy-carbonyl-, C₁-C₆-alkylamino-carbonyl- or di-(C₁-C₄-alkyl)-amino-carbonyl-substituted alkoxy, alkylamino or dialkylamino having in each case 1 to 8 carbon atoms in the alkyl groups, represents in each case optionally cyano- or halogen-substituted alkenyloxy, alkenylthio, alkenylamino, alkynyloxy, alkynylthio or alkynylamino having in each case 3 to 8 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 4 carbon atoms in the alkyl moiety, or represents in each case optionally nitro-, cyano-, hydroxyl-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted arylalkoxy, arylalkylthio or arylalkylamino having in each case 6 or 10 carbon atoms in the aryl group and 1 to 4 carbon atoms in the alkyl moiety, or together with R⁷ represents in each case optionally cyano-, hydroxy-, carboxyl-, carbamoyl-, thiocarbamoyl-, halogen-, C₁-C₆-alkyl-, C₁-C₆-haloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₁-C₆-alkoxy-carbonyl-, di-(C₁-C₄-alkyl)-amino-carbonyl- or di-(C₁-C₄-alkyl)-amino-sulfonyl-substituted thiaalkyleneoxy, thiaalkyleneamino, thiaalkenyleneamino, alkylenediamino or alkenylenediamino having in each case up to 5 carbon atoms, where in each case one methylene grouping is optionally replaced by O (oxygen), S (sulfur) or N (nitrogen),
A¹ represents a single bond or represents optionally hydroxyl-, cyano-, carboxyl-, phenyl-, C₁-C₆-alkyl-, C₁-C₆-haloalkyl-, C₁-C₆-alkoxy-, C₁-C₆-haloalkoxy- or C₁-C₆-alkoxy-carbonyl-substituted alkanediyl (alkylene) having 1 to 6 carbon atoms and
A² represents optionally hydroxyl-, cyano-, carboxyl-, C₁-C₆-alkyl-, C₁-C₆-haloalkyl-, C₁-C₆-alkoxy-, C₁-C₆-haloalkoxy- or C₁-C₆-alkoxy-carbonyl-substituted alkanediyl (alkylene) having 1 to 6 carbon atoms.

3. The compound of the formula (I) as claimed in claim 1 in which
R represents alkyl which has 1 to 4 carbon atoms and is mono- or polysubstituted by fluorine, chlorine and/or bromine,
Q¹ represents oxygen,
Q² represents the grouping 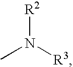
R¹ represents hydrogen, hydroxyl, in each case optionally cyano-, carboxyl-, carbamoyl-, halogen-, C₁-C₄-alkoxy- or C₁-C₄-alkoxy-carbonyl-substituted alkyl, alkylcarbonyl, alkoxy, alkoxycarbonyl, alkylcarbonyloxy or alkoxycarbonyloxy having in each case 1 to 6 carbon atoms in the alkyl groups, in each case optionally halogen-substituted alkylsulfonyl or alkylsulfonyloxy having in each case 1 to 6 carbon atoms, in each case optionally cyano-, carboxyl-, carbamoyl-, halogen- or C₁-C₄-alkoxy-carbonyl-substituted alkenyl, alkynyl, alkenyloxy or alkynyloxy having in each case 3 to 6 carbon atoms, in each case optionally cyano-, carboxyl-, carbamoyl-, halogen-, C₁-C₃-alkyl- or C₁-C₃-haloalkyl-substituted cycloalkyl, cycloalkyloxy, cycloalkylalkyl, cycloalkyl-alkoxy, cycloalkylcarbonyl, cycloalkylsulfonyl or cycloalkylsulfonyloxy having in each case 3 to 6 carbon atoms in the cycloalkyl group and, if appropriate, 1 to 4 carbon atoms in the alkyl moiety, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted aryl, aryloxy, arylalkyl, arylalkoxy, arylcarbonyl, arylsulfonyl or arylsulfonyloxy having in each case 6 or 10 carbon atoms in the aryl group and, if appropriate, 1 to 4 carbon atoms in the alkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted heterocyclyl, heterocyclylalkyl, heterocyclylcarbonyl, heterocyclylsulfonyl or heterocyclylsulfonyloxy having in each case up to 8 carbon atoms and at least one heteroatom from the group consisting of N (nitrogen), O (oxygen) or an S(O)ₙ grouping and optionally additionally a (C=Q¹) grouping in the heterocyclyl group and, if appropriate, 1 to 4 carbon atoms in the alkyl moiety,
R² represents one of the groupings -A¹-R⁶ or -A²-O—R⁶— or -A¹-S(O)ₙ—R⁶ or -A²-N(R⁶)₂ or -A²-N(R⁶)—O—R⁶ or -A²-O—N(R⁶)₂ or -A²-O—N═C(R⁶)₂ or -A²-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A²-(C=Q³)-N(R⁶)₂ or -A¹-(C=Q³)-N(R⁶)OR⁶ or -A²-O—(C=Q³)-R⁶ or -A²-N(R⁶)—(C=Q³)-R⁶, where the radicals R⁶—if a plurality of them is present in the same groupings—may be identical or different, or
R³ represents one of the groupings -A¹-R⁶ or -A¹-O—R⁶— or -A¹-S(O)ₙ—R⁶ or -A¹-N(R⁶)₂ or -A²-N(R⁶)—O—R⁶ or -A²-O—N(R⁶)₂ or -A²-O—N═C(R⁶)₂ or -A¹-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —N(R⁶)-A¹-(C=Q³)-R⁶ or -A¹-N(R⁶)—(C=Q³)-R⁶ or -A¹-C(R⁶)═N—O—R⁶ or -A¹-C(R⁶)═N(R⁶)—N(R⁶)₂ or A²-C(R⁶)═N(R⁶)—N(R⁶)(C=Q³)-(Q³)—R⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different,
R² and R³ together with the nitrogen atom which is attached to the two groups represent an optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted monocyclic or bicyclic heterocycle which contains up to 8 carbon atoms and optionally one to three further nitrogen atoms and/or one oxygen atom and/or one (SO)ₙ grouping and optionally additionally a (C=Q¹) grouping or, or
R² and R³ together represent the grouping ═P(X)₃, where X represents amino, C₁-C₄-alkyl, C₁-C₄-alkylamino, C₃-C₆-cycloalkyl, C₃-C₆-cycloalkylamino, phenyl or phenylamino and where the radicals X may in each case be identical or different, and
R⁴ represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkyl, alkoxy, alkylamino or dialkylamino having in each case 1 to 6 carbon atoms in the alkyl groups, in each case optionally cyano-, carboxyl-, carbamoyl-, thiocarbamoyl-, halogen-, C₁-C₄-alkoxy-carbonyl-, C₁-C₄-alkylamino-carbonyl- or di-(C₁-C₃-alkyl)-amino-carbonyl-substituted alkenyl or alkynyl having in each case 2 to 6 carbon atoms, in each case optionally cyano-, carboxyl-, carbamoyl-, halogen-, C₁-C₃-alkyl- or C₁-C₃-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate, 1 to 4 carbon atoms in the alkyl moiety, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl, di-(C₁-C₃-alkyl)-amino-carbonyl- or di-(C₁-C₃-alkyl)-amino-sulfonyl-substituted aryl, arylamino, N-alkyl-arylamino, arylalkyl, arylalkylamino, N-alkyl-arylalkylamino or N-cycloalkyl-arylalkylamino having in each case 6 or 10 carbon atoms in the aryl group and, if appropriate, 1 to 4 carbon atoms in the alkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted heterocyclyl or heterocyclylalkyl having in each case up to 8 carbon atoms and at least one heteroatom from the group consisting of N (nitrogen), O (oxygen) or an S(O)ₙ grouping and optionally additionally a (C═N—R¹) grouping in the heterocyclyl group and, if appropriate, 1 to 4 carbon atoms in the alkyl moiety,
R⁵ represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkyl, alkoxy, alkylamino or dialkylamino having in each case 1 to 6 carbon atoms in the alkyl groups, in each case optionally cyano-, carboxyl-, carbamoyl-, thiocarbamoyl-, halogen-, C₁-C₄-alkoxy-carbonyl-, C₁-C₄-alkylamino-carbonyl- or di-(C₁-C₃-alkyl)-amino-carbonyl-substituted alkenyl or alkynyl having in each case 2 to 6 carbon atoms, in each case optionally cyano-, carboxyl-, carbamoyl-, halogen-, C₁-C₃-alkyl- or C₁-C₃-haloalkyl-substituted cycloalkyl, cycloalkylamino, N-alkyl-cycloalkylamino, cycloalkenyl or cycloalkylalkyl having in each case 3 to 6 carbon atoms in the cycloalkyl group or cycloalkenyl group and, if appropriate, 1 to 4 carbon atoms in the alkyl moiety, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl, di-(C₁-C₃-alkyl)-amino-carbonyl- or di-(C₁-C₃-alkyl)-amino-sulfonyl-substituted aryl, arylamino, N-alkyl-arylamino, arylalkyl, arylalkylamino, N-alkyl-arylalkylamino or N-cycloalkyl-arylalkylamino having in each case 6 or 10 carbon atoms in the aryl group, if appropriate 1 to 4 carbon atoms in the alkyl moiety and if appropriate 3 to 6 carbon atoms in the cycloalkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted heterocyclyl or heterocyclylalkyl having in each case up to 8 carbon atoms and at least one heteroatom from the group consisting of N (nitrogen), O (oxygen) or an S(O)ₙ grouping in the heterocyclyl group and, if appropriate, 1 to 4 carbon atoms in the alkyl moiety, or together with R⁴ represents in each case optionally cyano-, halogen- or C₁-C₄-alkyl-substituted and/or benzo-fused alkanediyl (alkylene), alkyleneamino or alkylenediamino, each of which has 2 to 5 carbon atoms in the alkylene grouping and is optionally interrupted by O (oxygen) and/or N (nitrogen) and/or an S(O)ₙ grouping and/or a (C=Q¹) grouping,
R⁶ represents hydrogen, in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkyl, alkoxy, alkylamino, alkoxyamino, dialkylamino or N-alkyl-alkoxyamino having in each case 1 to 6 carbon atoms in the alkyl groups, in each case optionally cyano-, carboxyl-, carbamoyl-, thiocarbamoyl-, halogen-, C₁-C₄-alkoxy-carbonyl-, C₁-C₄-alkylamino-carbonyl- or di-(C₁-C₃-alkyl)-amino-carbonyl-substituted alkenyl, alkenyloxy, alkenylamino, N—C₁-C₄-alkyl-alkenylamino, alkynyl, alkynyloxy, alkynylamino or N—C₁-C₄-alkyl-alkynylamino having in each case 2 to 6 carbon atoms in the alkenyl or alkynyl groups, in each case optionally cyano-, carboxyl-, carbamoyl-, halogen-, C₁-C₃-alkyl- or C₁-C₃-haloalkyl-substituted cycloalkyl, cycloalkyloxy, cycloalkylamino, N-alkyl-cycloalkylamino, cycloalkylalkyl, cycloalkyl-alkoxy, cycloalkylalkylamino or N-alkyl-cycloalkylalkylamino having in each case 3 to 6 carbon atoms in the cycloalkyl groups and, if appropriate, 1 to 4 carbon atoms in the alkyl moiety, where the cycloalkyl groups each optionally contain a (C=Q¹) grouping, in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted aryl, aryloxy, arylthio, arylamino, N-alkyl-arylamino, arylalkyl, arylalkoxy, arylalkylthio, arylalkylamino or N-alkyl-arylalkylamino having in each case 6 or 10 carbon atoms in the aryl groups and, if appropriate, 1 to 4 carbon atoms in the alkyl moiety, or in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted monocyclic or bicyclic heterocyclyl or heterocyclylalkyl having in each case up to 8 carbon atoms and at least one hetero building block from the group consisting of N (nitrogen), O (oxygen), S(O)ₙ and optionally additionally a (C=Q³) grouping in the heterocyclyl group and, if appropriate, 1 to 4 carbon atoms in the alkyl moiety, or, if two groupings R⁶ are located in geminal position on a C═C double bond, these groupings together optionally also represent alkanediyl (divalent hydrocarbon chain, “alkylene”) having 4 or 5 carbon atoms, where one (hydro)carbon unit (C, CH, CH₂) may be replaced by O (oxygen), N (nitrogen), an S(O)ₙ grouping or a (C=Q¹) grouping,
R⁷ represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkoxy, alkylthio, alkylamino or dialkylamino having in each case 1 to 6 carbon atoms in the alkyl groups, represents in each case optionally cyano- or halogen-substituted alkenyloxy, alkenylthio, alkenylamino, alkynyloxy, alkynylthio or alkynylamino having in each case 3 to 6 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 3 carbon atoms in the alkyl moiety, or represents in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted phenylalkoxy, phenylalkylthio or phenylalkylamino having 1 to 3 carbon atoms in the alkyl moiety,
R⁸ represents in each case optionally cyano-, carboxyl-, carbamoyl-, thiocarbamoyl-, hydroxyl-, amino-, halogen-, C₁-C₄-alkoxy-, C₁-C₄-alkylamino-, di-(C₁-C₃-alkyl)-amino-, C₁-C₄-alkoxy-carbonyl-, C₁-C₄-alkylamino-carbonyl- or di-(C₁-C₃-alkyl)-amino-carbonyl-substituted alkoxy, alkylamino or dialkylamino having in each case 1 to 6 carbon atoms in the alkyl groups, represents in each case optionally cyano- or halogen-substituted alkenyloxy, alkenylthio, alkenylamino, alkynyloxy, alkynylthio or alkynylamino having in each case 3 to 6 carbon atoms, represents in each case optionally cyano-, halogen-, C₁-C₄-alkyl- or C₁-C₄-haloalkyl-substituted cycloalkyloxy, cycloalkylthio, cycloalkylamino, cycloalkylalkoxy, cycloalkylalkylthio or cycloalkylalkylamino having in each case 3 to 6 carbon atoms in the cycloalkyl group and, if appropriate, 1 to 3 carbon atoms in the alkyl moiety, or represents in each case optionally nitro-, cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted phenylalkoxy, phenylalkylthio or phenylalkylamino having 1 to 3 carbon atoms in the alkyl moiety, or together with R⁷ represents in each case optionally cyano-, hydroxyl-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, C₁-C₄-alkoxy-carbonyl-, di-(C₁-C₃-alkyl)-amino-carbonyl- or di-(C₁-C₃-alkyl)-amino-sulfonyl-substituted thiaalkyleneoxy, thiaalkyleneamino, thiaalkenyleneamino, alkylenediamino or alkenylenediamino having in each case up to 4 carbon atoms, where in each case one (hydro)carbon unit (C, CH, CH₂) is optionally replaced by O (oxygen), S (sulfur) or N (nitrogen),
A¹ represents a single bond or represents optionally hydroxyl-, cyano-, carboxyl-, phenyl-, C₁-C₄-alkyl-, C₁-C₄-haloalkyl-, C₁-C₄-alkoxy-, C₁-C₄-haloalkoxy- or C₁-C₄-alkoxy-carbonyl-substituted alkanediyl (alkylene) having 1 to 4 carbon atoms,
A² represents optionally hydroxyl-, cyano-, carboxyl-, C₁-C₄-alkyl-, C₁-C₄-haloalkyl-, C₁-C₄-alkoxy-, C₁-C₄-haloalkoxy- or C₁-C₄-alkoxy-carbonyl-substituted alkanediyl (alkylene) having 1 to 4 carbon atoms.

4. The compound of the formula (I) as claimed in claim 1 in which
R represents methyl, ethyl, n- or i-propyl, each of which is mono- or polysubstituted by fluorine and/or chlorine,
R¹ represents hydrogen, hydroxyl, in each case optionally cyano-, carboxyl-, carbamoyl-, fluorine-, chlorine-, methoxy-, ethoxy-, n- or i-propoxy-, methoxycarbonyl-, ethoxycarbonyl-, n- or i-propoxycarbonyl-substituted methyl, ethyl, n- or i-propyl, n-, i-, s- or t-butyl, acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, acetyloxy, propionyloxy, n- or i-butyroyloxy, methoxycarbonyloxy, ethoxycarbonyloxy, n- or i-propoxycarbonyloxy, in each case optionally fluorine-, chlorine- and/or bromine-substituted methylsulfonyl, ethylsulfonyl, n- or i-propylsulfonyl, n-, i-, s- or t-butyl-sulfonyl, methylsulfonyloxy, ethylsulfonyloxy, n- or i-propyl-sulfonyloxy, in each case optionally cyano-, fluorine-, chlorine-, bromine-, methoxycarbonyl-, ethoxycarbonyl-, n- or i-propoxycarbonyl-substituted propenyl, butenyl, propynyl, butynyl, propenyloxy, butenyloxy, propynyloxy or butynyloxy, in each case optionally cyano-, fluorine-, chlorine-, bromine-, methyl-, ethyl-, n- or i-propyl- or trifluoromethyl-substituted cyclopropyl, cyclobutyl, cyclopentyl, cyclohexyl, cyclopentyloxy, cyclohexyloxy, cyclopropylmethyl, cyclopentylmethyl, cyclohexylmethyl, cyclopentylmethoxy, cyclohexylmethoxy, cyclopropylcarbonyl, cyclobutylcarbonyl, cyclopentylcarbonyl, cyclopropyl-sulfonyl, cyclobutylsulfonyl, cyclopentylsulfonyl, cyclohexylsulfonyl, cyclopentylsulfonyloxy or cyclohexylsulfonyloxy, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, n- or i-propylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylamino-carbonyl- or dimethylaminosulfonyl-substituted phenyl, naphthyl, phenoxy, naphthyloxy, benzyl, phenylethyl, phenylmethoxy, phenylethoxy, benzoyl, naphthoyl, phenylsulfonyl, naphthylsulfonyl, phenylsulfonyloxy or naphthylsulfonyl-oxy, or in each case optionally nitro-, cyano-, hydroxyl-, carboxyl-, carbamoyl, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, n- or i-propylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted heterocyclyl, heterocyclylmethyl, heterocyclylsulfonyl or heterocyclylsulfonyloxy, where the heterocyclyl grouping is in each case selected from the group consisting of furyl, thienyl, pyrazolyl, pyridyl, pyrimidyl,
R² represents one of the groupings -A¹-R⁶ or -A²-O—R⁶— or -A¹-S(O)ₙ—R⁶ or -A²-N(R⁶)₂ or -A²-N(R⁶)—O—R⁶ or -A²-O—N(R⁶)₂ or -A²-O—N═C(R⁶)₂ or -A²-N(R⁶)—S(O)ₙ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A²-O—(C=Q³)-R⁶ or -A²-NH—(C=Q³)-R⁶, where the radical R⁶—if a plurality of them is present in the same groupings—may be identical or different,
R³ represents one of the groupings -A¹-R⁶ or -A¹-O—R⁶— or -A¹-S(O)ₙ—R⁶ or -A¹-N(R⁶)₂ or -A²-N(R⁶)—O—R⁶ or -A²-O—N(R⁶)₂ or -A²-O—N═C(R⁶)₂ or -A¹-N(R⁶)—S(O)ᵣ—R⁶ or -A²-N(R⁶)—N(R⁶)₂ or -A²-N(R⁶)—N═C(R⁶)₂ or -A¹-(C=Q³)-R⁶ or -A¹-(C=Q³)-Q³-R⁶ or -A¹-(C=Q³)-N(R⁶)₂ or -A¹-(C=Q³)-N(R⁶)OR⁶ or —O-A²-(C=Q³)-R⁶ or -A¹-O—(C=Q³)-R⁶ or —NH-A¹-(C=Q³)-R⁶ or -A¹-NH—(C=Q³)-R⁶ or -A¹-C(R⁶)═N—O—R⁶ or -A¹-C(R⁶)═N(R⁶)—N(R⁶)₂ or -A²-C(R⁶)═N(R⁶)—N(R⁶)—(C=Q¹)-(Q³)ₘR⁶ or -A²-C(R⁶)═N(R⁶)—N(R⁶)—(C=Q³)-N(R⁶)₂ or -A²-C(R⁶)═N(R⁶)—N(R⁶)—S(O)ₙ—R⁶, where the radicals R⁶—if a plurality of them is present in the same groupings—may be identical or different, or
R² and R³ represent the grouping ═CR⁷R⁸ or
R² and R³ together with the nitrogen atom to which the two groups are attached represent an optionally cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, trifluoromethoxy-, methylthio-, ethylthio-, n- or i-propylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted and optionally benzo-fused heterocycle from the group consisting of pyrrolidinyl, oxopyrrolidinyl, piperidinyl, oxopiperidinyl, morpholinyl, oxomorpholinyl and
R⁴ represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propylamino-carbonyl-, dimethylaminocarbonyl- or diethylaminocarbonyl-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino, n-, i-, s- or t-butylamino, dimethylamino or diethyl-amino, in each case optionally cyano-, fluorine-, chlorine-, bromine-, methoxycarbonyl-, ethoxycarbonyl-, n- or i-propoxycarbonyl-, methylaminocarbonyl-, ethylaminocarbonyl-, n- or i-propylamino-carbonyl-, dimethylaminocarbonyl- or diethylaminocarbonyl-substituted ethenyl, propenyl, butenyl, ethynyl, propynyl or butynyl, in each case optionally cyano-, fluorine-, chlorine-, bromine-, methyl-, ethyl- or trifluoromethyl-substituted cyclopropyl, cyclobutyl, cyclopentyl, cyclohexyl, cyclopropylamino, cyclobutylamino, cyclopentylamino, cyclohexylamino, N-methyl-cyclopropylamino, N-methyl-cyclobutylamino, N-methyl-cyclopentylamino, N-methyl-cyclohexylamino, cyclopentenyl, cyclohexenyl, cyclopropylmethyl, cyclobutylmethyl, cyclopentylmethyl or cyclohexylmethyl, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenyl, naphthyl, phenylamino, naphthylamino, N-methyl-phenylamino, N-ethyl-phenylamino, benzyl, phenylethyl, phenylmethylamino, phenylethylamino, N-methyl-phenylmethylamino, N-ethyl-phenylmethylamino or N-cyclopropyl-phenylmethylamino, or in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxy-carbonyl-, dimethylamino-carbonyl- or dimethylaminosulfonyl-substituted heterocyclyl or heterocyclylmethyl, where heterocyclyl is selected from the group consisting of furyl, tetrahydrofuryl, thienyl, pyrazolyl, pyrazolinyl, oxazolyl, oxazolinyl, isoxazolyl, thiazolyl, pyridyl, pyrimidyl,
R⁵ represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propylamino-carbonyl-, dimethylaminocarbonyl- or diethylaminocarbonyl-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino, n-, i-, s- or t-butylamino, dimethylamino or diethyl-amino, in each case optionally cyano-, fluorine-, chlorine-, bromine-, methoxycarbonyl-, ethoxycarbonyl-, n- or i-propoxycarbonyl-, methylaminocarbonyl-, ethylaminocarbonyl-, n- or i-propylamino-carbonyl-, dimethylaminocarbonyl- or diethylaminocarbonyl-substituted ethenyl, propenyl, butenyl, ethynyl, propynyl or butynyl, in each case optionally cyano-, fluorine-, chlorine-, bromine-, methyl-, ethyl- or trifluoromethyl-substituted cyclopropyl, cyclobutyl, cyclopentyl, cyclohexyl, cyclopropylamino, cyclobutylamino, cyclopentylamino, cyclohexylamino, cyclopentenyl, cyclohexenyl, cyclopropylmethyl, cyclobutylmethyl, cyclopentylmethyl or cyclohexylmethyl, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenyl, naphthyl, phenylamino, naphthylamino, N-methyl-phenylamino, N-ethyl-phenylamino, benzyl, phenylethyl, phenylmethylamino, phenylethylamino, N-methyl-phenylmethylamino, N-ethyl-phenylmethylamino or N-cyclopropyl-phenylmethylamino, or in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted heterocyclyl or heterocyclylmethyl, where heterocyclyl is selected from the group consisting of furyl, tetrahydrofuryl, thienyl, pyrazolyl, pyrazolinyl, oxazolyl, oxazolinyl, isoxazolyl, thiazolyl, pyridyl, pyrimidyl, or together with R⁴ represents in each case optionally cyano-, fluorine-, chlorine-, bromine-, methyl-, ethyl-, n- or i-propyl-substituted and/or benzo-fused propane-1,3-diyl, butane-1,4-diyl, pentane-1,5-diyl, 1-azapropane-1,3-diyl, 1-azabutane-1,4-diyl, 1-azapentane-1,5-diyl, 1,3-diazapropane-1,3-diyl, 1,4-diazabutane-1,4-diyl or 1,5-diazapentane-1,5-diyl, each of which is optionally interrupted by O, S, N, (C═O), (C═S), (SO) or (SO₂),
R⁶ represents hydrogen, in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxy-carbonyl-, methylaminocarbonyl-, ethylaminocarbonyl-, n- or i-propylaminocarbonyl-, dimethylaminocarbonyl- or diethylaminocarbonyl-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino, n-, i-, s- or t-butylamino, methoxyamino, ethoxyamino, n- or i-propoxyamino, n-, i-, s- or t-butoxyamino, dimethylamino, diethylamino, N-methyl-methoxyamino, N-ethyl-methoxyamino or N-methyl-ethoxy-amino, in each case optionally cyano-, fluorine-, chlorine-, bromine-, methoxycarbonyl-, ethoxycarbonyl-, n- or i-propoxycarbonyl-, methylaminocarbonyl-, ethylaminocarbonyl-, n- or i-propylaminocarbonyl-, dimethylaminocarbonyl- or diethylaminocarbonyl-substituted ethenyl, propenyl, butenyl, propenyloxy, butenyloxy, propenylamino, butenylamino, N-methyl-propenylamino, N-ethyl-propenylamino, N-methyl-butenylamino, N-ethyl-butenylamino, ethynyl, propynyl, butynyl, propynyl-oxy, butynyloxy, propynylamino, butynylamino, N-methyl-propynylamino, N-ethyl-propynylamino, N-methyl-butynylamino or N-ethyl-butynylamino, in each case optionally cyano-, fluorine-, chlorine-, bromine-, methyl-, ethyl- or trifluoromethyl-substituted cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, N-methyl-cyclopropylamino, N-methyl-cyclobutylamino, N-methyl-cyclopentylamino, N-methyl-cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethylamino, cyclobutylmethylamino, cyclopentylmethylamino, cyclohexylmethylamino, N-methyl-cyclopropylmethylamino, N-methyl-cyclobutylmethylamino, N-methyl-cyclopentyl-methylamino or N-methyl-cyclohexylmethylamino, in each case optionally nitro-, cyano-, hydroxyl-, carboxyl-, carbamoyl-, thiocarbamoyl-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxy-carbonyl-, dimethylaminocarbonyl- or dimethylaminosulfonyl-substituted phenyl, naphthyl, phenoxy, naphthyloxy, phenylthio, naphthylthio, phenylamino, naphthylamino, N-methyl-phenylamino, benzyl, phenylmethyl, phenyl-ethyl, phenylmethoxy, phenylethoxy, phenylmethylthio, phenylethylthio, phenylmethylamino, phenylethylamino or N-methyl-phenylmethylamino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted heterocyclyl or heterocyclylmethyl, where the heterocyclyl grouping is selected from the group consisting of oxetanyl, furyl, tetrahydrofuryl, oxo-tetrahydrofuryl, thienyl, tetrahydrothienyl, oxo- and dioxo-tetrahydrothienyl, pyrrolyl, pyrrolinyl, pyrrolidinyl, oxopyrrolidinyl, dioxopyrrolidinyl, pyrazolyl, pyrazolinyl, imidazolyl, imidazolinyl, oxazolyl, oxazolinyl, thiazolyl, thiazolinyl, oxadiazolyl, thiadiazolyl, tetrahydropyranyl, oxotetrahydropyranyl, benzotetrahydropyranyl, tetrahydrothiopyranyl, oxo- and dioxotetrahydrothiopyranyl, dioxacyclohexyl, piperidinyl, oxo-piperidinyl, morpholinyl, oxomorpholinyl, pyridinyl, pyrimidinyl,
R⁷ represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propylaminocarbonyl-, dimethylaminocarbonyl- or diethylaminocarbonyl-substituted methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino or dipropylamino, represents in each case optionally cyano-, fluorine-, chlorine- or bromine-substituted propenyloxy, butenyloxy, pentenyloxy, propenylthio, butenylthio, pentenylthio, propenylamino, butenylamino, pentenylamino, propynyloxy, butynyloxy, pentynyloxy, propynylthio, butynylthio, pentynylthio, propynylamino, butynylamino or pentynylamino, represents in each case optionally cyano-, fluorine-, chlorine-, bromine-, methyl-, ethyl-, n- or i-propyl- or trifluoromethyl-substituted cyclopentyloxy, cyclohexyloxy, cyclopentylthio, cyclohexylthio, cyclopentylamino, cyclohexylamino, cyclopentylmethoxy, cyclohexylmethoxy, cyclopentylmethylthio, cyclohexylmethylthio, cyclopentylmethylamino or cyclohexylmethylamino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenylmethoxy, phenylmethylthio or phenylmethylamino,
R⁸ represents in each case optionally cyano-, fluorine-, chlorine-, bromine-, methoxy-, ethoxy-, n- or i-propoxy-, methylamino-, ethylamino-, n- or i-propylamino-, dimethylamino-, diethylamino-, methoxycarbonyl-, ethoxycarbonyl-, n- or i-propoxycarbonyl-, methylaminocarbonyl-, ethylaminocarbonyl-, n- or i-propylaminocarbonyl-, dimethylaminocarbonyl- or diethylaminocarbonyl-substituted methoxy, ethoxy, n- or i-propoxy, n-, i-, s- or t-butoxy, methylamino, ethylamino, n- or i-propylamino, n-, i-, s- or t-butyl-amino, dimethylamino, diethylamino or dipropylamino, represents in each case optionally cyano-, fluorine-, chlorine- or bromine-substituted propenyloxy, butenyloxy, pentenyloxy, propenylthio, butenylthio, pentenylthio, propenylamino, butenylamino, pentenylamino, propynyloxy, butynyloxy, pentynyloxy, propynylthio, butynylthio, pentynylthio, propynylamino, butynylamino or pentynylamino, represents in each case optionally cyano-, fluorine-, chlorine-, bromine-, methyl-, ethyl-, n- or i-propyl- or trifluoromethyl-substituted cyclopentyloxy, cyclohexyloxy, cyclopentylthio, cyclohexyl-thio, cyclopentylamino, cyclohexylamino, cyclopentylmethoxy, cyclohexylmethoxy, cyclopentylmethylthio, cyclohexylmethylthio, cyclopentylmethylamino or cyclohexylmethylamino, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, dimethylaminocarbonyl- or dimethylaminosulfonyl-substituted phenylmethoxy, phenylmethylthio or phenylmethylamino, or together with R⁷ represents in each case optionally cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy-, trifluoromethoxy-, methylthio-, ethylthio-, n- or i-propylthio-, difluoromethylthio-, trifluoromethylthio-, methylsulfinyl-, ethylsulfinyl-, trifluoromethylsulfinyl-, methylsulfonyl-, ethylsulfonyl-, trifluoromethylsulfonyl-, methoxycarbonyl-, ethoxycarbonyl-, n- or i-propoxycarbonyl-, methylaminocarbonyl-, ethylaminocarbonyl-, n- or i-propylamino-carbonyl-, dimethylamino-carbonyl- or dimethylaminosulfonyl-substituted thiaethyleneoxy, thiapropyleneoxy, thiaethyleneamino, thiapropyleneamino, ethylene-diamino or propylendiamino,
A¹ represents a single bond, represents methylene, or represents in each case optionally hydroxyl-, cyano-, carboxyl-, methyl-, ethyl-, n- or i-propyl-, trifluoromethyl-, methoxy-, ethoxy-, n- or i-propoxy-, trifluoromethoxy-, methoxycarbonyl-, ethoxycarbonyl-, n- or i-propoxycarbonyl-substituted ethane-1,1-diyl, ethane-1,2-diyl, propane-1,1-diyl, propane-1,2-diyl or propane-1,3-diyl,
A² represents methylene or represents in each case optionally hydroxyl-, cyano-, carboxyl-, methyl-, ethyl-, n- or i-propyl-, trifluoromethyl-, methoxy-, ethoxy-, n- or i-propoxy-, trifluoromethoxy-, methoxycarbonyl-, ethoxycarbonyl-, n- or i-propoxy-carbonyl-substituted ethane-1,1-diyl, ethane-1,2-diyl, propane-1,1-diyl, propane-1,2-diyl or propane-1,3-diyl.

5. The compound of the formula (I) as claimed in claim 1 in which R represents CF₃ and the radicals Q¹ and Q² are as defined in claim 3.

6. The compound of the formula (I) as claimed in claim 1 in which R represents CF₃, Q¹ represents oxygen, Q² represents the grouping 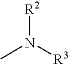
and R² represents hydrogen and R³ is as defined in claim 3.

7. The compound of the formula (I) as claimed in claim 1 in which R represents CF₃, Q¹ represents oxygen, R³ represents CH₂CN, Q² represents the grouping 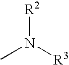
and R² is as defined in claim 3.

8. The compound of the formula (I) as claimed in claim 1 in which R represents CF₃, Q¹ represents oxygen, Q² represents the grouping 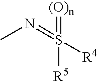
and R⁴, R⁵ and n are as defined in claim 3.

9. A compound of the formula (II), 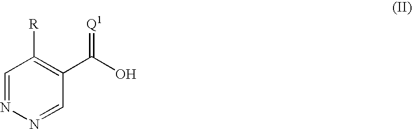
in which R and Q¹ are as defined in claim 1.

10. A compound of the formula (IIc) 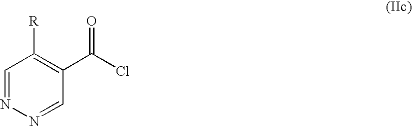
in which R is as defined in claim 1.

11. A process for preparing compounds of the formula (I) as claimed in claim 1, characterized in that pyridazinecarboxylic acids of the formula (II) or reactive derivatives thereof 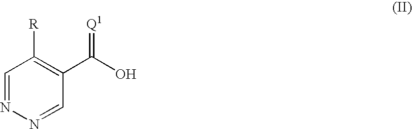
in which R and Q¹ are as defined in claim 1 are reacted with nitrogen compounds of the general formula (III),
H-Q² (III)
in which Q² is as defined in claim 1, if appropriate in the presence of one or more reaction auxiliaries and if appropriate in the presence of one or more diluents and the resulting compounds of the formula (I) are, if appropriate, subjected to further reactions according to customary methods to convert them into other compounds of the formula (I) according to the above definitions. 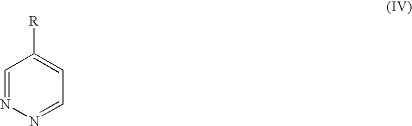
in which R is as defined in claim 1,
is reacted in a first reaction step with methyl pyruvate of the formula (V) 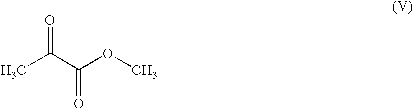
in the presence of hydrogen peroxide, in the presence of an acid, if appropriate in the presence of a further reaction auxiliary and in the presence of one or more diluents, and worked up in a customary manner, and the resulting novel pyridazinecarboxylic esters of the formula (IIa) 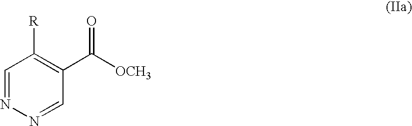
in which R is as defined in claim 1 are, in a second reaction step, hydrolyzed and worked up in a customary manner.

12. A composition for controlling pests and/or for treating plants, which composition comprises at least one compound of the formula (I) as claimed in claim 1, in addition to extenders and/or surfactants.

13. The use of compounds of the formula (I) as claimed in claim 1 for controlling pests and/or for treating plants.

14. A method for controlling pests and/or for treating plants, characterized in that compounds of the formula (I) as claimed in claim 1 are applied to the unwanted microorganisms and/or their habitat.

15. A process for preparing compositions as claimed in claim 6, characterized in that compounds of the formula (I) as claimed in claim 1 are mixed with extenders and/or surfactants.
